(19) United States
(12) Patent Application Publication
(10) Pub. No.: US 2023/0133064 A1
(43) Pub. Date: May 4, 2023

(54) T CELL MANUFACTURING PROCESS

(71) Applicant: Adaptimmune Limited, Abingdon (GB)

(72) Inventor: Katerina Mardilovich, Abingdon (GB)

(21) Appl. No.: 18/046,874

(22) Filed: Oct. 14, 2022

Related U.S. Application Data
(63) Continuation of application No. PCT/GB2021/050909, filed on Apr. 16, 2021.

(30) Foreign Application Priority Data
Apr. 17, 2020 (GB) 2005617.2

(51) Int. Cl. C12N 5/0783; C07K 14/725

(52) U.S. Cl. CPC C12N 5/0636; C07K 14/7051; C12N 2501/00

(57) ABSTRACT

The present invention relates to improved methods for manufacturing T cells and improved T cell compositions resulting therefrom.

### CROSS-REFERENCE TO RELATED APPLICATIONS

This application is a continuation of International Patent Application No. PCT/GB2021/050909, filed Apr. 16, 2021, which claims the benefit of GB Application No. 2005617.2, filed Apr. 17, 2020, the content of each of which is incorporated herein by reference in its entirety.

### REFERENCE TO AN ELECTRONIC SEQUENCE LISTING

The contents of the electronic sequence listing (364761.xml; Size: 61,440 bytes; and Date of Creation: Oct. 14, 2022) is herein incorporated by reference in its entirety.

### TECHNICAL FIELD

The present invention relates to improved methods for manufacturing T cells and improved T cell compositions resulting therefrom. The invention further relates to methods for T cell manufacturing that provide improved T cell expansion and result in a T cell population with improved persistence, memory function, and antigen stimulated survival. The invention further relates to the use of the improved T cell population in adoptive T cell immunotherapies including adoptive therapy for the treatment of tumour and cancer.

### BACKGROUND

Adoptive cell therapy (ACT) can comprise the intravenous transfer of tumour-resident peripheral blood modified immune cells into cancer patients to mediate an anti-tumour function and thereby offers the opportunity to treat diseases including cancer, infectious disease, autoimmune disease, inflammatory disease, and immunodeficiency. ACT may also comprise the transfer of tumour-infiltrating lymphocytes (TILs) or natural killer cells (NK cells) as a basis for cell therapy of cancer. Additionally ACT using gene-modified T cells expressing novel T cell receptors (TCR) or chimeric antigen receptors (CAR) offers the opportunity to provide large pools of tumour specific T cells that can be generated, with specific and potent anti-tumour activity for improved and targeted clinical responses recognising specific tumour expressed antigens.

ACT processes typically involve a step of donor subject leukapheresis in which the blood of the donor is passed through an apparatus that separates out the white blood cells from the sample of blood. The isolated T cell population then are subjected to processes including activation and expansion steps, and optionally genetic modification to introduce specific CAR or TCR molecules to generate the required active T cell numbers for a therapeutic dose to be clinically effective.

Current T cell manufacturing processes often require multiple rounds of activation and expansion to achieve a large enough T cell population for a therapeutic dose. Expansion of the population is a limiting step and the T cell activation and expansion methods often result in a cell population which is high in the more differentiated subsets of cells; these cells are by their nature more limited in survival being short lived and hence provide a short lived and less potent anti-tumour activity and memory function. Such T cell populations are prone to exhaustion and loss of effector immune cell function and suffer with a detriment to in-vivo expansion of the transferred T cells and lack persistence after infusion into patients.

The present invention addresses the above described limitations of current processes of T cell manufacturing and therapeutic T cell and provides methods therapeutic T cell compositions of improved expansion, persistence and survival.

### SUMMARY OF THE INVENTION

The present invention generally provides an improved method of producing T cells and/or populations of T cells and thereby provides an improved population of T cells and/or composition of T cells made by the method, the invention further provides the use of the improved population of T cells and/or composition of T cells in adoptive therapy for treatment of disease including cancer, infectious disease, autoimmune disease, inflammatory disease, and immunodeficiency. The method according to the invention provides improved T cell expansion, survival duration, effector and memory function in-vitro and/or in-vivo.

According to the present invention there is provided a method of manufacturing modified T cells and/or population of modified T cells comprising the steps of:

(a) activating an isolated population of T cells,

(b) culturing the T cells,

(c) modifying the T cells to express a heterologous T cell receptor (TCR) or chimeric antigen receptor (CAR),

(d) adding an inhibitor of AKT (AKT inhibitor) to the modified T cells,

(e) culturing the modified T cells or T cell population to expand and/or proliferate the cells,

(f) optionally harvesting and/or cryopreserving the modified T cells or T cell population; optionally wherein the heterologous TCR or CAR binds or specifically binds to a cancer and/or tumour antigen or peptide antigen thereof; optionally wherein the T cells are modified to express a heterologous T cell receptor (TCR) or chimeric antigen receptor (CAR), for example by transducing the T cells with a nucleic acid or vector comprising a nucleic acid encoding one or more heterologous T cell receptor (TCR) and/or one or more chimeric antigen receptor (CAR).

T Cell Adoptive Therapy

The isolated T cells or population of T cells may be T cells, tumour infiltrating cytotoxic T lymphocytes (TILs), or natural killer cells (NK cells). The T cells can be, Natural Killer T (NKT) cells, or precursors thereof including embryonic stem cells, and pluripotent stem cells (e.g, those from which lymphoid cells may be differentiated). The T cells can be lymphocytes that mature in the thymus and are chiefly responsible for cell-mediated immunity and also involved in the adaptive immune system. According to the present invention the T cells can include, but are not limited to, helper T cells, cytotoxic T cells, memory T cells (including central memory T cells, stem-cell-like memory T cells (or stem-like memory T cells), or memory T cells: e.g. , TEM cells and TEMRA cells, Regulatory T cells (also known as suppressor T cells), Natural killer T cells, Mucosal associated invariant T cells, or gamma-delta T cells. Cytotoxic T cells (CTL or killer T cells) are a subset of T-lymphocytes capable of inducing the death of infected somatic or tumour cells. T cells provided according to the invention herein may be CD8⁺ T cells or CD4⁺ T cells; or CD4⁺ T cells and CD8⁺ T cells. For example, the T cells may be a mixed population of CD4⁺ T cells and CD8⁺ T cells. CD4⁺ T cells are known as T helper cells (T_{H} cells) express the CD4 surface glycoprotein and play an important role in the adaptive immune system, helping the activity of other immune cells by releasing T cell cytokines and helping to suppress or regulate immune responses. They are essential for the activation and growth of cytotoxic T cells. CD8⁺ T cells are known as Cytotoxic T cells (T_{C} cells, CTLs, killer T cells) and express the CD8 surface glycoprotein. CD8⁺ T cells act to destroy virus-infected cells and tumour cells. Most CD8⁺ T cells express TCRs that can recognise a specific antigen displayed on the surface of infected or damaged cells by a class I MHC molecule. Specific binding of the TCR and optionally CD8 glycoprotein to the antigen and MHC molecule leads to T cell-mediated destruction of the infected or damaged cells.

The isolated population of T cells may be isolated from a donor subject. According to the invention the T cells produced by the method of the invention may be used for adoptive cellular therapy or adoptive immunotherapy. The donor subject and the recipient individual (receiving adoptive cellular therapy or adoptive immunotherapy) may be the same (i.e. autologous treatment; the T cells are obtained from an individual who is subsequently treated with the modified T cells) or the donor individual and the recipient individual may be different (i.e. allogeneic treatment; the T cells are obtained from one individual and subsequently used to treat a different individual). Autologous refers to any material derived from a subject to which it is later to be re-introduced into the same subject.

Accordingly adoptive cellular therapy or adoptive immunotherapy in the context of the present invention refers to the adoptive transfer of the isolated T cells or population of T cells that are engineered by gene transfer to express genetically modified TCRs or CARs and/or co-receptors (e.g. CD8), specific for surface antigens, antigen peptides or antigen peptide expressed on target cells, optionally expressed thereon as MHC complexes. This can be used to treat a range of diseases depending upon the target chosen, e.g., tumour or cancer specific antigens or antigen peptides so as to treat cancer or tumour. In brief, the adoptive cellular therapy involves removing a portion of a donor's or the patient's white blood cells using a process called leukapheresis. The T cells, TILs or NK cells may then be expanded and mixed with expression vectors comprising the TCR/CAR polynucleotide and/or co-receptor (e.g. CD8), in order to transfer the TCR/CAR and/or co-receptor (e.g. CD8) to the T cells, TILS or NK cells. The T cells, TILS or NK cells are expanded again and at the end of the expansion, the engineered T cells or NK cells may be washed, concentrated, and then frozen to allow time for testing, shipping and storage until a patient is ready to receive the infusion of engineered cells.

Cell Culture

The modified T cells may be cultured using any convenient means, technique, vessel, container or system to produce the expanded population. Suitable culture systems include stirred tank fermenters, airlift fermenters, roller bottles, culture bags or dishes, and other bioreactors, in particular hollow fibre bioreactors. Preferably the use of such systems is well-known in the art. Preferably the culture means is a gas permeable rapid expansion culture, for example G-Rex™, a device for expansion or static expansion of cells such as non-adherent cells. And or cells according to the methods of the invention.

Cell Modification

According to the invention the T cells are modified to express one or more heterologous T cell receptor (TCR) and/or one or more chimeric antigen receptor (CAR), modification may be by transducing the T cells with a nucleic acid or vector comprising a nucleic acid encoding the one or more heterologous T cell receptor (TCR) and/or the one or more chimeric antigen receptor (CAR). The T cells may also be modified by incorporation of a nucleic acid encoding the one or more heterologous T cell receptor (TCR) and/or the one or more chimeric antigen receptor (CAR) into the genome of the T cell, for example into the genome of a progenitor to a T cell, for example an induced pleuripotent stem cell or lymphoid lineage cell derived therefrom for example mature T cell derived therefrom.

According to the invention the modified T cells may be modified to comprise a heterologous nucleic acid or nucleic acid construct or vector comprising the nucleic acid or construct, encoding a heterologous T cell receptor (TCR) or heterologous chimeric antigen receptor (CAR). Optionally the TCR may be an affinity enhanced TCR, for example a specific peptide enhanced affinity receptor (SPEAR) TCR.

According to the present invention the method may comprise the inclusion of a poloxamer at any stage or step within the process, preferably at the modification or transduction of the T cells or T cell population, preferably to aid modification and/or transduction, optionally at a level of up to or about one half of the multiplicity of infection (MOI) of virus with respect to cell concentration (i.e. 0.5 virus per cell), optionally any one of between 0.1-0.2, 0.3-0.4, 0.5-0.6 0.6-0.7, 0.7-0.8, 0.8-0.9, 0.9-1.0, 1.0-1.1, 1.1-1.2, 1.2-1.3, 1.3-1.4, 1.4-1.5, 1.5-1.6, 1.6-1.7, 1.7-1.8, 1.8-1.9, 1.9-2.0.MOI.

Heterologous TCR/CAR

According to the present invention the modified T cells can express at least one heterologous T cell receptor (TCR) and/or heterologous chimeric antigen receptor (CAR) which binds or specifically binds to a cancer and/or tumour antigen or peptide antigen thereof, preferably wherein the peptide antigen is associated with a cancerous condition, cancer and/or tumour and/or is presented by tumour of cancer cell or tissue, optionally presented by HLA/MHC. Upon binding to the antigen, the modified T cells or population of T cells can exhibit T cell effector functions and/or cytolytic effects towards cells bearing the antigen and/or undergo proliferation and/or cell division. In certain embodiments, the modified T cells or population of T cells comprising the TCR exhibits comparable or better therapeutic potency compared to cells comprising a chimeric antigen receptor (CAR) targeting the same cancer and/or tumour antigen and/or peptide (antigenic peptide). The activated modified T cells or population of T cells comprising the heterologous TCR or CAR can secrete anti-tumor cytokines which can include, but are not limited to, TNFalpha, IFNγ and IL2.

The term “heterologous” or “exogenous” refers to a polypeptide or nucleic acid that is foreign to a particular biological system, such as a cell or host cell, and is not naturally present in that system and which may be introduced to the system by artificial or recombinant means. Accordingly, the expression of a TCR or CAR which is heterologous, may thereby alter the immunogenic specificity of the T cells so that they recognise or display improved recognition for one or more cancer and/or tumour antigen or peptide antigen thereof that are present on the surface of cancer cells of an individual with cancer. The modification of T cells and their subsequent expansion may be performed in vitro and/or ex vivo.

According to the present invention the cancer and/or tumour antigen or peptide antigen thereof may be a cancer-testis antigen, NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (α-fetoprotein), MAGE-A1, MAGE-A2, MAGE-A3, MAGE-A4, MAGE-A6, MAGE-A8, and MAGE-A9, MAGE-A10, or MAGE-A12, or peptide antigen thereof. Preferably the tumour antigen is MAGE-A4 or AFP or peptide antigen thereof. Preferably the cancer and/or tumour antigen peptide is a peptide antigen of MAGE A4 and/or comprises the amino acid sequence GVYDGREHTV, SEQ ID NO: 1 (MAGE A4) or is a peptide antigen of alpha fetoprotein (AFP) and/or comprises the sequence FMNKFIYEI (SEQ ID No: 2) or residues 158-166 derived from alpha fetoprotein (AFP) (SEQ ID NO: 3).

According to the invention the heterologous TCR or CAR binds or specifically binds to a cancer and/or tumour antigen or peptide antigen thereof associated with a tumour or a cancerous condition and/or presented by tumour or cancer cell or tissue and/or binds or specifically binds to tumour cells and/or tissue and/or cancer cells and/or tissue of a subject, patient or cancer patient suffering from a disease condition or cancerous condition. The subject, patient or cancer patient may be subsequently treated with the modified T cells or population thereof according to the invention. Suitable cancer patients for treatment according to the invention with the modified T cells may be identified by a method comprising; obtaining sample of tumour and/or cancer cells from an individual or subject with tumour and/or cancer and; identifying the cancer cells as binding to the heterologous TCR or CAR expressed.

Specificity describes the strength of binding between the heterologous TCR or CAR and a specific target cancer and/or tumour antigen or peptide antigen thereof and may be described by a dissociation constant, Kd, the ratio between bound and unbound states for the receptor-ligand system. Additionally, the fewer different cancer and/or tumour antigens or peptide antigen thereof the heterologous TCR or CAR can bind, the greater its binding specificity. According to the invention the heterologous TCR or CAR may bind to less than 10, 9, 8, 7, 6, 5, 4, 3, 2 different cancer and/or tumour antigens or peptide antigen thereof.

According to the invention the heterologous TCR or CAR may bind with a dissociation constant of between , 0.01 μM and 100 μM, between 0.01 μM and 50 μM, between 0.01 μM and 20 μM, between 0.05 μM and 20 μM or of 0.01, 0.02, 0.03, 0.04, 0.05, 0.06, 0.07, 0.08, 0.09, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.75 μM, 0.8 μM, 0.85 μM, 0.9 μM, 0.95 μM, 1.0 μM, 1.5 μM, 2.0 μM, 2.5 μM, 3.0 μM, 4.0 μM, 4.5 μM, 5.0 μM, 5.5 μM, 6.0 μM, 6.5 μM, 7.0 μM, 7.5 μM, 8.0 μM, 8.5 μM, 9.5 μM, 10.0 μM; or between 10 μM and 1000 μM, between 10 μM and 500 μM, between 50 μM and 500 μM or of 10, 20 30, 40, 50 60, 70, 80, 90, 100 μM, 150 μM, 200 μM, 250 μM, 300 μM, 350 μM, 400 μM, 450 μM, 500 μM; optionally measured with surface plasmon resonance, optionally at 25° C., optionally between a pH of 6.5 and 6.9 or 7.0 and 7.5. The dissociation constant, K_{D} or k_{off}/kₒₙ may be determined by experimentally measuring the dissociation rate constant, k_{off}, and the association rate constant, kₒₙ. A TCR dissociation constant may be measured using a soluble form of the TCR, wherein the TCR comprises a TCR alpha chain variable domain and a TCR beta chain variable domain.

Accordingly, a heterologous TCR or CAR for use in accordance with the invention is capable of binding efficiently and/or with high affinity to a cancer and/or tumour antigen or peptide antigen of MAGE A4 or AFP, preferably a peptide comprising GVYDGREHTV, SEQ ID NO: 1 (MAGE A4 peptide) or FMNKFIYEI (AFP peptide) SEQ ID NO: 2, optionally in complex with a peptide presenting molecule for example an HLA, for example with HLA-A*02, optionally selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642, alternatively without presentation in complex with a peptide presenting molecule, for example HLA, for example with a dissociation constant of between 0.01 μM and 100 μM such as 50 μM, 100 μM, 200 μM, 500 μM, preferably between 0.05 μM to 20.0 μM. For example the heterologous TCR or CAR may have the property of binding to an endogenously expressed tumour cell surface a cancer and/or tumour antigen or peptide antigen thereof optionally wherein the binding is independent of presentation of the cell surface antigen as a complex with an peptide-presenting or antigen-presenting molecule, for example major histocompatibility complex (MHC) or human leukocyte antigen (HLA) or major histocompatibility complex class related protein (MR)1.

According to the present invention the TCR or CAR binding may be specific for one cancer and/or tumour antigen, for example a MAGE protein such as MAGE A4 or AFP, or peptide antigen thereof optionally in comparison to a closely related cancer and/or tumour antigen or peptide antigen sequence. The closely related cancer and/or tumour antigen or peptide antigen sequence may be of similar or identical length and/or may have a similar number or identical number of amino acid residues. The closely related peptide antigen sequence may share between 50 or 60 or 70 or 80 to 90% identity, preferably between 80 to 90% identity and/or may differ by 1, 2, 3 or 4 amino acid residues. The closely related peptide sequence may be derived from a sequence comprising the polypeptide sequence of sequence GVYDGREHTV (SEQ ID NO: 1) or FMNKFIYEI (SEQ ID NO: 2). The binding affinity may be determined by equilibrium methods (e.g. enzyme-linked immunosorbent assay (ELISA) or radioimmunoassay (RIA)), or kinetics (e.g. BIACORE™ analysis). Avidity is the sum total of the strength of binding of two molecules to one another at multiple sites, e.g. taking into account the valency of the interaction. According to the invention the immunoresponsive cells may demonstrate improved affinity and/or avidity to a cancer and/or tumour antigen or peptide antigen thereof, or a cancer and/or tumour antigen or peptide antigen thereof presented by tumour of cancer cell or tissue and recognised by the heterologous TCR or CAR in comparison in comparison to T cells lacking the heterologous TCR or CAR or having an alternative heterologous TCR or CAR.

According to the invention, the heterologous TCR or CAR may selectively bind to a cancer and/or tumour antigen or peptide antigen thereof as herein before described, optionally associated with a cancerous condition and/or presented by tumour of cancer cell or tissue; optionally wherein the cancer and/or tumour antigen or peptide antigen thereof is recognised by the heterologous TCR or CAR, optionally in complex with a peptide presenting molecule for example an HLA, alternatively without presentation in complex with a peptide presenting molecule or HLA, preferably expressed by a tumour cell or a cancer cell or tissue. Selective binding denotes that the heterologous TCR or CAR binds with greater affinity to one cancer and/or tumour antigen or peptide antigen thereof in comparison to another. Selective binding is denoted by the equilibrium constant for the displacement by one ligand antigen of another ligand antigen in a complex with the heterologous TCR or CAR.

According to the present invention the heterologous TCR or CAR binding is selective and/or specific for a cancer and/or tumour antigen or peptide antigen as herein described. According to the present invention the heterologous TCR or CAR may bind and/or bind specifically and/or bind selectively a peptide presenting molecule for example an HLA presenting or displaying a cancer and/or tumour antigen or peptide antigen thereof, i.e. a peptide fragment of a cancer and/or tumour antigen (pHLA), wherein the HLA corresponds to MHC class I (A, B, and C) which all are the HLA Class1 or specific alleles thereof or the HLA corresponds to MHC class II (DP, DM, DO, DQ, and DR) or specific alleles thereof, preferably the HLA is class 1, preferably the allele is HLA-A2 or HLA-A*02 or an HLA-A2+or HLA-A*02 positive HLA, optionally selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642. Alternatively, the heterologous TCR or CAR may bind and/or bind specifically and/or bind selectively a cancer and/or tumour antigen or peptide antigen thereof, which is not presented or displayed by a peptide presenting molecule, for example HLA.

Heterologous TCR

Preferably, the heterologous TCR or CAR is not naturally expressed by the immunoresponsive cells (i.e. the TCR or CAR is exogenous or heterologous). A heterologous TCR may include αβTCR heterodimers. A heterologous TCR or CAR may be a recombinant or synthetic or artificial TCR or CAR i.e. a TCR that does not exist in nature. For example, a heterologous TCR may be engineered to increase its affinity or avidity for a specific cancer and/or tumour antigen or peptide antigen thereof (i.e. an affinity enhanced TCR or specific peptide enhanced affinity receptor (SPEAR) TCR). The affinity enhanced TCR or (SPEAR) TCR may comprise one or more mutations relative to a naturally occurring TCR, for example, one or more mutations in the hypervariable complementarity determining regions (CDRs) of the variable regions of the TCR α and β chains. These mutations may increase the affinity of the TCR for MHCs that display a peptide fragment of a tumour antigen optionally when expressed by tumour and/or cancer cells. Suitable methods of generating affinity enhanced or matured TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to tumour or cancer cells expressing the tumour antigen of the MAGE family, for example MAGE A4 or peptide antigen thereof for example the sequence GVYDGREHTV, SEQ ID NO: 1 or a peptide antigen of alpha fetoprotein (AFP) and/or comprises the sequence FMNKFIYEI (SEQ ID No: 2) or residues 158-166 derived from alpha fetoprotein (AFP) (SEQ ID NO: 3).

According to the invention the heterologous TCR may be a MAGE A4 TCR which may comprise the α chain reference amino acid sequence of SEQ ID NO: 4 or a variant thereof and the β chain reference amino acid sequence of SEQ NO: 6 or a variant thereof.

According to an alternative embodiment, the heterologous TCR may be an AFP TCR which may comprise the α chain reference amino acid sequence of SEQ ID NO: 16 or a variant thereof and the β chain reference amino acid sequence of SEQ NO: 18 or a variant thereof.

A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence.

The TCR may be encoded by the α chain reference nucleotide sequence of SEQ ID NO: 5 or a variant thereof and the β chain reference nucleotide sequence of SEQ NO: 7 or a variant thereof.

According to the alternative embodiment, the TCR may be encoded by the α chain reference nucleotide sequence of SEQ ID NO: 17 or a variant thereof and the β chain reference nucleotide sequence of SEQ NO: 19 or a variant thereof.

A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence.

According to the present invention the TCR (MAGE A4 TCR) may comprise a TCR alpha chain variable domain and a TCR beta chain variable domain, wherein:
(i) the alpha chain variable domain comprises CDRs having the sequences

VSPFSN (αCDR1), SEQ ID NO:10 or amino acids 48-53 of SEQ ID NO:4,

LTFSEN (αCDR2), SEQ ID NO:11 or amino acids 71-76 of SEQ ID NO:4, and

CVVSGGTDSWGKLQF (αCDR3), SEQ ID NO:12 or amino acids 111-125 of SEQ ID NO:4, and
(ii) the beta chain variable domain comprises CDRs having the sequences

KGHDR (βCDR1), SEQ ID NO:13 or amino acids 46-50 of SEQ ID NO:6,

SFDVKD (βCDR2), SEQ ID NO:14 or amino acids 68-73 of SEQ ID NO:6, and

CATSGQGAYEEQFF (βCDR3), SEQ ID NO:15 or amino acids 110-123 of SEQ ID NO:6; or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto, optionally 100% sequence identity thereto.

According to the alternative embodiment, the TCR (AFP TCR) may comprise a TCR alpha chain variable domain and a TCR beta chain variable domain, wherein:
(i) the alpha chain variable domain comprises CDRs having the sequences

DRGSQS (αCDR1), SEQ ID NO:22 or amino acids 27-32 of SEQ ID NO:16,

IYSNGD (αCDR2), SEQ ID NO:23or amino acids 50-55 of SEQ ID NO:16, and

AVNSDSGYALNF (αCDR3), SEQ ID NO:24 or amino acids 90-101 of SEQ ID NO:16, and
(ii) the beta chain variable domain comprises CDRs having the sequences

SGDLS (βCDR1), SEQ ID NO:25 or amino acids 27-31 of SEQ ID NO:18,

YYNGEE (βCDR2), SEQ ID NO:26 or amino acids 49-54 of SEQ ID NO:18, and

ASSLGGESEQY (βCDR3), SEQ ID NO:27 or amino acids 92-102 of SEQ ID NO:18; or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto, optionally 100% sequence identity thereto.

According to the alternative embodiment the TCR may have substituted:

a. αCDR1 having the sequence DRGSQA, SEQ ID NO:28,

b. αCDR2 having the sequence AVNSDSSYALNF, SEQ ID NO:29,

c. αCDR2 having the sequence AVNSDSGVALNF, SEQ ID NO:30,

d. αCDR1 having the sequence DRGSQA, SEQ ID NO:28 and αCDR2 having the sequence AVNSDSGVALNF, SEQ ID NO:30,

e. αCDR2 having the sequence AVNSQSGYALNF, SEQ ID NO: 31,

f. αCDR2 having the sequence AVNSQSGYSLNF, SEQ ID NO: 32,

g. αCDR2 having the sequence AVNSQSSYALNF, SEQ ID NO: 36

h. αCDR1 having the sequence DRGSQA, SEQ ID NO:28 and αCDR2 having the sequence AVNSQSGYALNF, SEQ ID NO: 31,

i. αCDR2 having the sequence AVNSQSGVALNF, SEQ ID NO: 32,

j. αCDR2 having the sequence AVNSQNGYALNF, SEQ ID NO: 33,

k. αCDR1 having the sequence DRGSFS, SEQ ID NO: 34,

l. αCDR1 having the sequence DRGSYS, SEQ ID NO: 35,

m. αCDR1 having the sequence DRGSYS, SEQ ID NO: 35 and αCDR2 having the sequence AVNSDSSYALNF SEQ ID NO: 29,

n. αCDR1 having the sequence DRGSYS, SEQ ID NO: 35 and αCDR2 having the sequence AVNSDSSYALNF SEQ ID NO: 29,

o. αCDR1 having the sequence DRGSYS, SEQ ID NO: 35 and αCDR2 having the sequence AVNSQSGYALNF, SEQ ID NO: 31.

Accordingly, the TCR (MAGE A4 TCR) may comprise a TCR in which the alpha chain variable domain comprises an amino acid sequence that has at least 70%, 75%, 80%, 85%, 90%, 95%, 96%, 97%, 98% or 99% identity to SEQ ID NO:8 or the sequence of amino acid residues 1-136 of SEQ ID NO:4, and/or the beta chain variable domain comprising an amino acid sequence that has at least 70%, 75%, 80%, 85%, 90%, 95%, 96%, 97%, 98% or 99% identity to SEQ ID NO:9 or the sequence of amino acid residues 1-133 of SEQ ID NO:6.

Alternatively, the TCR (AFP TCR) may comprise a TCR in which the alpha chain variable domain comprises an amino acid sequence that has at least 70%, 75%, 80%, 85%, 90%, 95%, 96%, 97%, 98% or 99% identity to SEQ ID NO:20 or the sequence of amino acid residues 1-112 of SEQ ID NO:16, and/or the beta chain variable domain comprising an amino acid sequence that has at least 70%, 75%, 80%, 85%, 90%, 95%, 96%, 97%, 98% or 99% identity to SEQ ID NO:21 or the sequence of amino acid residues 1-112 of SEQ ID NO:18.

The terms “progenitor TCR” or “parental TCR”, is used herein to refer to a TCR comprising the MAGE A4 TCR α chain and MAGE A4 TCR β chain of SEQ ID NOs: 4 and 6 respectively or alternatively a TCR comprising the AFP TCR α chain and AFP TCR β chain of SEQ ID NOs: 16 and 18 respectively. It is desirable to provide TCRs that are mutated or modified relative to the progenitor TCR that have an equal, equivalent or higher affinity and/or an equal, equivalent or slower off-rate for the peptide-HLA complex than the progenitor TCR. According to the invention the heterologous TCR may have more than one mutation present in the alpha chain variable domain and/or the beta chain variable domain relative to the progenitor TCR and may be denoted, “engineered TCR” or “mutant TCR”. These mutation(s) may improve the binding affinity and/or specificity and/or selectivity and/or avidity for MAGE A4 or peptide antigen thereof. In certain embodiments, there are 1, 2, 3, 4, 5, 6, 7 or 8 mutations in alpha chain variable domain, for example 4 or 8 mutations, and/or 1, 2, 3, 4 or 5 mutations in the beta chain variable domain, for example 5 mutations. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 8 for MAGE A4 TCR, SEQ ID NO: 20 for AFP TCR. In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 9 for MAGE A4 TCR or SEQ ID NO: 21 for AFP TCR.

According to the invention the TCR may comprise a TCR (MAGE A4 TCR) in which, the alpha chain variable domain comprises SEQ ID NO: 8 or the amino acid sequence of amino acid residues 1-136 of SEQ ID NO:4, or an amino acid sequence in which amino acid residues 1-47, 54-70, 77-110 and 126-136 thereof have at least 70%, 75%, 80%, 85%, 90%, 95%, 96%, 97%, 98% or 99% identity to the sequence of amino acid residues 1-47, 54-70, 77-110 and 126-136 respectively of SEQ ID NO:8 and/or in which amino acid residues 48-53, 71-76 and 111-125, CDR 1, CDR 2, CDR 3 respectively, have at least 70%, 75%, 80%, 85%, 90%, 95%, 96%, 97%, 98% or 99% identity to the sequence of amino acid residues 48-53, 71-76 and 111-125, CDR 1, CDR 2, CDR 3, respectively of SEQ ID NO:8.

According to the invention, the TCR (MAGE A4) may comprise a TCR in which, in the alpha chain variable domain, the sequence of:
(i) amino acid residues 1-47 thereof may have (a) at least 70%, 75%, 80%, 85%, 90% or 95% identity to the sequence of amino acid residues 1-47 of SEQ ID NO:8 or (b) may have one, two or three amino acid residues inserted or deleted relative to residues 1-47 of SEQ ID NO:8,
(ii) amino acid residues 48-53 is VSPFSN, CDR 1, SEQ ID NO:10 or amino acids 48-53 of SEQ ID NO:8,
(iii) amino acid residues 54-70 thereof may have (a) at least 70%, 75%, 80%, 85%, 90% or 95% identity to the sequence of amino acid residues 54-70 of SEQ ID NO: 25 or (b) may have one, two or three amino acid residues inserted or deleted relative to the sequence of amino acid residues 54-70 of SEQ ID NO: 8,
(iv) amino acid residues 71-76 may be LTFSEN, CDR 2, SEQ ID NO:11 or amino acids 71-76 of SEQ ID NO:8,
(v) amino acid residues 77-110 thereof may have at least 70%, 75%, 80%, 85%, 90% or 95% identity to the sequence of amino acid residues 77-110 of SEQ ID NO:8 or may have one, two or three insertions, deletions or substitutions relative to the sequence of amino acid residues 77-110 of SEQ ID NO:8,
(vi) amino acids 111-125 may be CVVSGGTDSWGKLQF, CDR 3, SEQ ID NO:12 or amino acids 111-125 of SEQ ID NO:8,
(vii) amino acid residues 126-136 thereof may have at least 70%, 75%, 80%, 85%, 90% or 95% identity to the sequence of amino acid residues 126-136 of SEQ ID NO: 8 or may have one, two or three insertions, deletions or substitutions relative to the sequence of amino acid residues 126-136 of SEQ ID NO:8.

According to the invention, the TCR may comprise a TCR, or MAGE A4 TCR, in which, in the beta chain variable domain comprises the amino acid sequence of SEQ ID NO:9, or an amino acid sequence in which amino acid residues 1-45, 51-67, 74-109, 124-133 thereof have at least 70%, 75%, 80%, 85%, 90% or 95% identity to the sequence of amino acid residues 1-45, 51-67, 74-109, 124-133 respectively of SEQ ID NO:9 and in which amino acid residues 46-50, 68-73 and 110-123 have at least 70%, 75%, 80%, 85%, 90% or 95% identity to the sequence of amino acid residues 46-50, 68-73 and 110-123, CDR 1, CDR 2, CDR 3, respectively of SEQ ID NO:9.

According to the invention, the TCR may comprise a TCR in which, in the beta chain variable domain, the sequence of:
(i) amino acid residues 1-45 thereof may have (a) at least 70%, 75%, 80%, 85%, 90% or 95% identity to the sequence of amino acid residues 1-45 of SEQ ID NO:9 or (b) may have one, two or three amino acid residues inserted or deleted relative to residues 1-45 of SEQ ID NO:9,
(ii) amino acid residues 46-50 is KGHDR, CDR 1, SEQ ID NO:13 or amino acids 46-50 of SEQ ID NO:9,
(iii) amino acid residues 51-67 thereof may have (a) at least 70%, 75%, 80%, 85%, 90% or 95% identity to the sequence of amino acid residues 51-67 of SEQ ID NO:9 or (b) may have one, two or three amino acid residues inserted or deleted relative to the sequence of amino acid residues 51-67 of SEQ ID NO:9,
(iv) amino acid residues 68-73 may be SFDVKD, CDR 2, SEQ ID NO:14 or amino acids 68-73 of SEQ ID NO:9,
(v) amino acid residues 74-109 thereof may have at least 70%, 75%, 80%, 85%, 90% or 95% identity to the sequence of amino acid residues 74-109 of SEQ ID NO:9 or may have one, two or three insertions, deletions or substitutions relative to the sequence of amino acid residues 74-109 of SEQ ID NO:9;
(vi) amino acids 110-123 may be CATSGQGAYEEQFF, CDR 3, SEQ ID NO:15 or amino acids 110-123 of SEQ ID NO:9,
(vii) amino acid residues 124-133 thereof may have at least 70%, 75%, 80%, 85%, 90% or 95% identity to the sequence of amino acid residues 124-133 of SEQ ID NO:9 or may have one, two or three insertions, deletions or substitutions relative to the sequence of amino acid residues 124-133 of SEQ ID NO:9.

In the alternative the TCR may comprise a TCR, or AFP TCR, in which, the alpha chain variable domain comprises the amino acid sequence of amino acid residues 1-112 of SEQ ID NO:16, or an amino acid sequence in which amino acid residues 1-26, 33-49, 56-89 and 102-112 thereof have at least 70%, 75%, 80%, 85%, 90%, 95%, 96%, 97%, 98% or 99% identity to the sequence of amino acid residues 1-26, 33-49, 56-89 and 102-112 respectively of SEQ ID NO:16 and/or in which amino acid residues 27-32, 50-55, 90-101, CDR 1, CDR 2, CDR 3 respectively, have at least 70%, 75%, 80%, 85%, 90%, 95%, 96%, 97%, 98% or 99% identity to the sequence of amino acid residues 27-32, 50-55, 90-101, CDR 1,

CDR 2, CDR 3, respectively of SEQ ID NO:16.

According to the invention, the TCR may comprise a TCR in which, in the alpha chain variable domain, the sequence of:
(i) amino acid residues 1-26 thereof may have (a) at least 70%, 75%, 80%, 85%, 90% or 95% identity to the sequence of amino acid residues 1-26 of SEQ ID NO:16 or (b) may have one, two or three amino acid residues inserted or deleted relative to residues 1-26 of SEQ ID NO:16,
(ii) amino acid residues 27-32 is DRGSQS, αCDR 1, SEQ ID NO:22 or amino acids 27-32 of SEQ ID NO:16,
(iii) amino acid residues 33-49 thereof may have (a) at least 70%, 75%, 80%, 85%, 90% or 95% identity to the sequence of amino acid residues 33-49 of SEQ ID NO: 16 or (b) may have one, two or three amino acid residues inserted or deleted relative to the sequence of amino acid residues 33-49 of SEQ ID NO: 16,
(iv) amino acid residues 50-55 may be IYSNGD, αCDR 2, SEQ ID NO:23 or amino acids 50-55 of SEQ ID NO:16,
(v) amino acid residues 56-89 thereof may have at least 70%, 75%, 80%, 85%, 90% or 95% identity to the sequence of amino acid residues 56-89 of SEQ ID NO:16 or may have one, two or three insertions, deletions or substitutions relative to the sequence of amino acid residues 56-89 of SEQ ID
(vi) amino acids 90-101 may be AVNSDSGYALNF, αCDR 3, SEQ ID NO:24 or amino acids 90-101 of SEQ ID NO:16,
(vii) amino acid residues 102-112 thereof may have at least 70%, 75%, 80%, 85%, 90% or 95% identity to the sequence of amino acid residues 102-112 of SEQ ID NO: 16 or may have one, two or three insertions, deletions or substitutions relative to the sequence of amino acid residues 102-112 of SEQ ID NO: 16.

According to the invention, the TCR, or AFP TCR, may comprise a TCR in which, in the beta chain variable domain comprises the amino acid sequence of amino acid residues 1-112 of SEQ ID NO:18, or an amino acid sequence in which amino acid residues 1-26, 32-48, 55-91, 103-112 thereof have at least 70%, 75%, 80%, 85%, 90% or 95% identity to the sequence of amino acid residues 1-26, 32-48, 55-91, 103-112 respectively of SEQ ID NO:18 and in which amino acid residues 27-31, 49-54 and 92-102 have at least 70%, 75%, 80%, 85%, 90% or 95% identity to the sequence of amino acid residues 27-31, 49-54 and 92-102, βCDR 1, βCDR 2, βCDR 3, respectively of SEQ ID NO:18.

According to the invention, the TCR may comprise a TCR in which, in the beta chain variable domain, the sequence of:
(i) amino acid residues 1-26 thereof may have (a) at least 70%, 75%, 80%, 85%, 90% or 95% identity to the sequence of amino acid residues 1-26 of SEQ ID NO:18 or (b) may have one, two or three amino acid residues inserted or deleted relative to residues 1-26 of SEQ ID NO:18,
(ii) amino acid residues 27-31 is SGDLS, βCDR 1, SEQ ID NO:25 or amino acids 27-31 of SEQ ID NO:18,
(iii) amino acid residues 32-48 thereof may have (a) at least 70%, 75%, 80%, 85%, 90% or 95% identity to the sequence of amino acid residues 32-48 of SEQ ID NO:18 or (b) may have one, two or three amino acid residues inserted or deleted relative to the sequence of amino acid residues 32-48 of SEQ ID NO:18,
(iv) amino acid residues 49-54 may be YYNGEE, βCDR 2, SEQ ID NO:26 or amino acids 49-54 of SEQ ID NO:18,
(v) amino acid residues 55-91 thereof may have at least 70%, 75%, 80%, 85%, 90% or 95% identity to the sequence of amino acid residues 55-91 of SEQ ID NO:18 or may have one, two or three insertions, deletions or substitutions relative to the sequence of amino acid residues 55-91 of SEQ ID NO:18
(vi) amino acids 92-102 may be ASSLGGESEQY, βCDR 3, SEQ ID NO:27 or amino acids 92-102 of SEQ ID NO:18
(vii) amino acid residues 103-112 thereof may have at least 70%, 75%, 80%, 85%, 90% or 95% identity to the sequence of amino acid residues 103-112 of SEQ ID NO:18 or may have one, two or three insertions, deletions or substitutions relative to the sequence of amino acid residues 103-112 of SEQ ID NO:18.

Accordingly the heterologous TCR may comprise a TCR in which the alpha chain comprises amino acid residues of SEQ ID No: 20, and the beta chain variable domain comprises amino acid residues of SEQ ID No: 21 or SEQ ID NO:42.

Co-Receptor Modified T Cells

According to the present invention, the population of modified T cells expressing or presenting a heterologous TCR or CAR may further express or present a heterologous co-receptor. The heterologous co-receptor may be a CD8 co-receptor. The CD8 co-receptor may comprise a dimer or pair of CD8 chains which comprises a CD8-α and CD8-β chain or a CD8-α and CD8-α chain. Preferably, the CD8 co-receptor is a CD8αα co-receptor comprising a CD8-α and CD8-α chain. A CD8α co-receptor may comprise the amino acid sequence of at least 80% identity to SEQ ID NO: 37, SEQ ID NO: 37 or a variant thereof. The CD8α co-receptor may be a homodimer.

The CD8 co-receptor binds to class 1 MHCs and potentiates TCR signalling. According to the invention the CD8 co-receptor may comprise the reference amino acid sequence of SEQ ID NO: 37 or may be a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence SEQ ID NO: 37. The CD8 co-receptor may be encoded by the reference nucleotide sequence of SEQ ID NO: 38 or may be a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence SEQ ID NO: 38.

According to the invention the heterologous CD8 co-receptor may comprise a CD8 co-receptor in which, in the Ig like V-type domain comprises CDRs having the sequence;
(i) VLLSNPTSG, CDR1, SEQ ID NO: 39, or amino acids 45-53 of SEQ ID NO: 37,
(ii) YLSQNKPK, CDR2, SEQ ID NO: 40 or amino acids 72-79 of SEQ ID NO: 37,
(iii) LSNSIM, CDR3, SEQ ID NO: 41 or amino acids 80-117 of SEQ ID NO: 37,
   or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto.

According to the invention the heterologous CD8 co-receptor may comprise a CD8 co-receptor which comprises or in which, in the Ig like V-type domain comprises, residues 22-135 of the amino acid sequence of SEQ ID No:37, or an amino acid sequence in which amino acid residues 22-44, 54-71, 80-117, 124-135 thereof have at least 70%, 75%, 80%, 85%, 90% or 95% identity to the sequence of amino acid residues 22-44, 54-71, 80-117, 124-135, CDR 1, CDR 2, CDR 3, respectively of SEQ ID No:37 and in which amino acid residues 45-53, 72-79 and 118-123 have at least 70%, 75%, 80%, 85%, 90% or 95% identity to the sequence of amino acid residues 45-53, 72-79 and 118-123 respectively of SEQ ID No:37.

According to the invention the CD8 co-receptor may comprise a CD8 co-receptor in which, or in which in the Ig like V-type domain, the sequence of:
(i) amino acid residues 22-44 thereof may have (a) at least 70%, 75%, 80%, 85%, 90% or 95% identity to the sequence of amino acid residues 22-44 of SEQ ID NO:37 or (b) may have one, two or three amino acid residues inserted or deleted relative to residues 22-44 of SEQ ID NO:37,
(ii) amino acid residues 45-53 is VLLSNPTSG, SEQ ID NO:39, CDR1, or amino acids 45-53 of SEQ ID NO:37,
(iii) amino acid residues 54-71 thereof may have (a) at least 70%, 75%, 80%, 85%, 90% or 95% identity to the sequence of amino acid residues 54-71 of SEQ ID NO:37 or (b) may have one, two or three amino acid residues inserted or deleted relative to the sequence of amino acid residues 54-71 of SEQ ID NO:37,
(iv) amino acid residues 72-79 may be YLSQNKPK, CDR2, SEQ ID NO:40 or amino acids 72-79 of SEQ ID NO:37,
(v) amino acid residues 80-117 thereof may have at least 70%, 75%, 80%, 85%, 90% or 95% identity to the sequence of amino acid residues 80-117 of SEQ ID NO:37 or may have one, two or three insertions, deletions or substitutions relative to the sequence of amino acid residues 80-117 of SEQ ID NO:37;
(vi) amino acids 118-123 may be LSNSIM, CDR3, SEQ ID NO:41 or amino acids 80-117 of SEQ ID NO:37,
(vii) amino acid residues 124-135 thereof may have at least 70%, 75%, 80%, 85%, 90% or 95% identity to the sequence of amino acid residues 124-135 of SEQ ID NO:37 or may have one, two or three insertions, deletions or substitutions relative to the sequence of amino acid residues 124-135 of SEQ ID NO:37.

The modified T cells that express heterologous CD8 co-receptor may demonstrate improved affinity and/or avidity and/or improved T-cell activation, as determinable by the assays disclosed herein, towards or on stimulation by antigenic peptide, tumour or cancer antigen optionally when presented on HLA relative to modified T cells that do not express heterologous CD8 co-receptor. The heterologous CD8 of modified T cells may interact or bind specifically to an MHC, the MHC may be class I or class II, preferably class I major histocompatibility complex (MHC), HLA-I molecule or with the MHC class I HLA-A/B2M dimer, preferably the CD8-α interacts with the α₃ portion of the Class I MHC (between residues 223 and 229), preferably via the IgV-like domain of CD8. Accordingly the heterologous CD8 improves TCR binding of the T cells to the HLA and/or antigenic peptide bound or presented by HLA pMHCI or pHLA, optionally on the surface of antigen presenting cell, dendritic cell and/or tumour or cancer cell, tumour or cancer tissue compared to T cells lacking the heterologous CD8.

Accordingly the heterologous CD8 can improve or increase the off-rate (k_{off}) of the cell (TCR)/peptide-major histocompatibility complex class I (pMHCI) interaction of the immunoresponsive cells, and hence its half-life, optionally on the surface of antigen presenting cell, dendritic cell and/or tumour or cancer cell, or tumour or cancer tissue compared to the cells lacking the heterologous CD8, and thereby may also provide improved ligation affinity and/or avidity. The heterologous CD8 can improve organizing the TCR on the immunoresponsive cell surface to enable cooperativity in pHLA binding and may provide improved therapeutic avidity. Accordingly, the heterologous CD8 co-receptor modified T cells may bind or interact with LCK (lymphocyte-specific protein tyrosine kinase) in a zinc-dependent manner leading to activation of transcription factors like NFAT, NF-κB, and AP-1. Heterologous CD8 modified T cells may have an improved or increased expression of CD40L, cytokine production, cytotoxic activity, induction of dendritic cell maturation or induction of dendritic cell cytokine production, optionally in response to cancer and/or tumour antigen or peptide antigen thereof optionally as presented by tumour of cancer cell or tissue, in comparison to T cells lacking the heterologous CD8 co-receptor.

Costimulatory Ligand Modified T Cells

According to the present invention, a modified T cell or a population of modified T cells may further comprise and/or express at least one exogenous and/or recombinant co-stimulatory ligand, optionally one, two, three or four. The interaction between the TCR and at least one exogenous co-stimulatory ligand may provide a non-antigen-specific signal and activation of the cell. Co-stimulatory ligands include, but are not limited to, members of the tumour necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, but are not limited to, nerve growth factor (NGF), CD40L (CD40L)/CD154, CD137L/4-1BBL, TNF-alpha, CD134L/OX40L/CD252, CD27L/CD70, Fas ligand (FasL),CD30L/CD153, tumour necrosis factor beta (TNFP)/lymphotoxin-alpha (LTa),lymphotoxin-beta (TTb), CD257/B cell-activating factor (BAFF)/Blys/THANK/Ta11-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28. In certain embodiments, the at least one co-stimulatory ligand is selected from the group consisting of 4-1BBL, CD275, CD80, CD86, CD70, OX40L, CD48, TNFRSF14, and combinations thereof. According to the present invention the modified T cell or a population of modified T cells may further comprise at least one exogenous and/or recombinant co-stimulatory ligand can be 4-1BBL or CD80, preferably 4-1BBL, alternatively 4-1BBL and CD80.

CD3+ Enrichment

According the present invention the method may further comprise the step wherein the T cells are enriched for CD3+ fraction, for T cells expressing CD3, an antigen, cluster of differentiation protein and part of the T cell receptor (TCR) complex on a mature T lymphocyte. Enrichment may be performed before modification of the T cells. Enrichment may be performed before activation of the T cells. Alternatively, and preferably enrichment is performed either during or after activation of the T cells, preferably either during or after activation of the T cells and before modification. Optionally enrichment is performed on T cells comprised on anti-CD3 antibody or antigen binding fragment thereof and/or anti-CD28 antibody or antigen binding fragment thereof, optionally attached to a removable bead for example as during activation optionally before modification.

T Cell Activation

According to the method of the present invention the activation or the activating the T cells or population of T cells stimulates the T cells to proliferate and/or expand.

Activating an isolated population of T cells may be achieved by a variety of methods, for example by contacting the T cells with an anti-CD3 antibody or CD3-binding fragment thereof, or contacting the T cells with an anti-CD28 antibody or a CD28-binding fragment thereof, or contacting the T cells with a B7 protein, (B7 is a type of peripheral membrane protein found on activated antigen-presenting cells that can produce a costimulatory signal, when paired with either a CD28 or CD152 (CTLA-4) surface protein on a T cell) or a CD28-binding fragment thereof, for example B7-1 or B7-2 or a CD28-binding fragment thereof. The activation means may be attached to a solid and optionally removeable surface or substrate such as a bead or magnetic bead, for example a magnetic bead coated with anti-CD3 and/or anti-CD28. Preferably the activation is by addition of anti-CD3 antibody or antigen binding fragment thereof and/or anti-CD28 antibody or antigen binding fragment thereof, optionally attached to a bead, optionally a removable bead, for example which may be a magnetic bead and therefore capable of isolation from the cell culture medium. T cell activation can be performed simultaneously with or after T cell modification, simultaneously with or after AKTi addition, simultaneously with or after both T cell modification and AKTi addition. Preferably T cell activation is prior to T cell modification, preferably prior to AKTi addition, preferably prior to both T cell modification and AKTi addition.

AKT Inhibitor Step

According to the method of the present invention the T cell modification or transduction can be performed prior to or simultaneously with activation, for example any one of about or greater than about 0, 1, 2, 3, 4, 5, 6, 7, 8, 9, 10, 11, 12, 13, 14, 15, 16, 17, 18, 19, or 20 hours prior to activation.

According to the method of the present invention the T cell modification or transduction can be performed after activation, preferably 18-26 hours after activation, preferably any of between 12-40, 13-38, 14-36, 15-34, 16-32, 17-30, 18-28, 18-26 or 18 to 24 hours after activation or any one of about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 hours after activation.

According to the method of the present invention the AKT inhibitor can be added after the T cell modification or transduction, preferably the AKT inhibitor can be added any one of between 8-42, 9-41, 10-40, 11-39, 12-38, 13-37, 14-36, 15-35, 16-34, 17-33, 18-32, 19-31, 20-30, 21-29, 22-28, 23-27, 24-26 or 24-25 hours after modification or transduction, preferably any one of between, 15-26, 16-25, 17-24, 18-23, 19-22, or 20-21, preferably 17-24, hours after T cell modification or transduction.

According to the method of the present invention the AKT inhibitor can be added after the T cell activation preferably the AKT inhibitor can be added any one of between 35-50, 36-49, 37-48, 38-47, 39-46, 40-45, 41-44, 42-43, hours after the T cell activation, preferably 35-50, hours after T cell modification or transduction.

AKT Inhibitors

According to the method of the present invention the AKT inhibitor can be selected from the group consisting of: an allosteric inhibitor or allosteric AKT inhibitor, a competitive ATP inhibitor, an inhibitor of interaction between AKT and the phospholipids, an inhibitor of phosphorylation of molecules downstream of AKT preferably of phosphorylation of PRAS 40, Ribosomal S6, or TSC2. The AKT inhibitor can also be selected from the group consisting of: an inhibitor of DNA-PK activation of AKT, an inhibitor of PDK-1 activation of AKT, an inhibitor of mTORC2 activation of AKT, an inhibitor of HSP activation of AKT.

Accordingly the allosteric inhibitor or allosteric AKT inhibitor may be selected from any one of:

(a) ARQ092, Miransertib, CAS No.: 1313881-70-7, formula C₂₇H₂₄N₆, or compound with structure

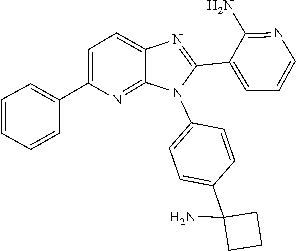 [SMILES: Nc1ncccc1-c1nc2ccc(-c3ccccc3)nc2n1-c1ccc(C2(N)CCC2)cc1]

(b) ARQ751, or compound with structure

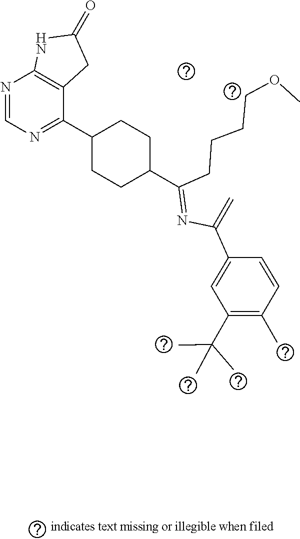 [SMILES: C=C(/N=C(\CCCCOC)C1CCC(c2ncnc3c2CC(=O)N3)CC1)c1ccc(C)c(C(C)(C)C)c1]

(c) BAY1125976, (CAS No. 1402608-02-9), formula C₂₃H₂₁N₅₀, or compound with structure

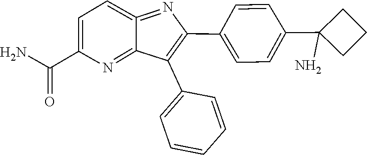

or

(d) MK-2206, (CAS No. 1032350-13-2), formula C₂₅H₂₁N₅O, or compound with structure

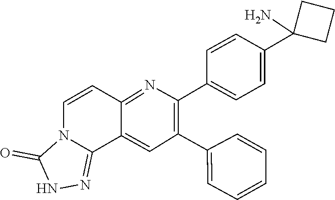 [SMILES: NC1(c2ccc(-c3nc4ccn5c(=O)[nH]nc5c4cc3-c3ccccc3)cc2)CCC1]

Accordingly the competitive ATP inhibitor can be selected from any one of:

(a) Afuresertib (GSK2110183), (CAS No: 1047645-82-8), formula C₁₈H₁₇Cl₂FN₄OS.HCl, or compound with structure

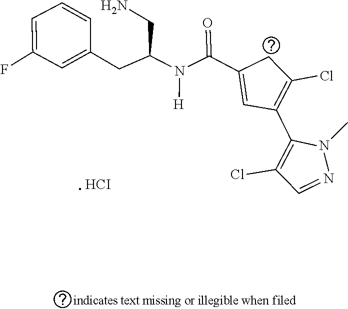

(b) Uprosertib, GSK2141795, (CAS Nbo: 1047634-65-0), formula C₁₈H₁₆Cl₂F₂N₄O₂, or compound with structure

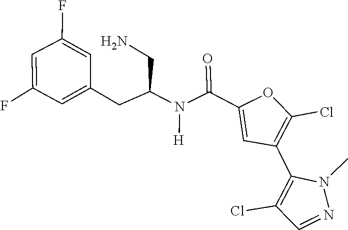 [SMILES: Cn1ncc(Cl)c1-c1cc(C(=O)N[C@H](CN)Cc2cc(F)cc(F)c2)oc1Cl]

(c) GSK690693, (CAS No: 937174-76-0), formula C₂₁H₂₇N₇O₃, or compound with structure

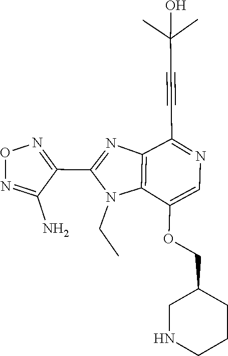 [SMILES: CCn1c(-c2nonc2N)nc2c(C#CC(C)(C)O)ncc(OC[C@H]3CCCNC3)c21]

(d) Ipatasertib (GDC-0068), (CAS No:1001264-89-6), formula C₂₄H₃₂ClN₅O₂, or compound with structure

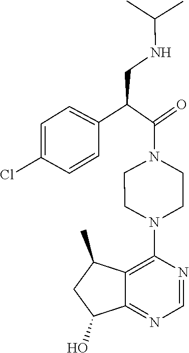 [SMILES: CC(C)NC[C@@H](C(=O)N1CCN(c2ncnc3c2[C@H](C)C[C@H]3O)CC1)c1ccc(Cl)cc1]

(e) LY2780301, or compound with structure

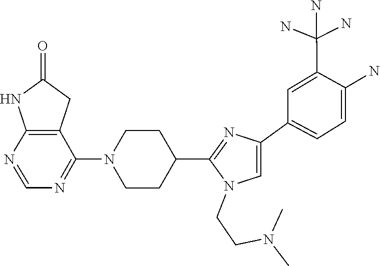 [SMILES: CN(C)CCn1cc(-c2ccc(N)c(C(N)(N)N)c2)nc1C1CCN(c2ncnc3c2CC(=O)N3)CC1]

(f) Triciribine (TCN-PM; VD-0002), (CAS No: 35943-35-2), formula C₁₃H₁₆N₆O₄, or compound with structure

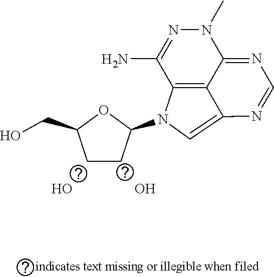

(g) AZD5363, (CAS 1143532-39-1), formula C₂₁H₂₅ClN₆O₂, or compound with structure

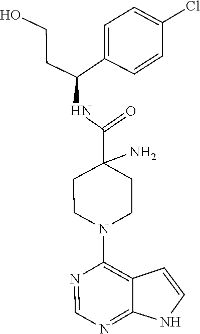 [SMILES: NC1(C(=O)N[C@@H](CCO)c2ccc(Cl)cc2)CCN(c2ncnc3[nH]ccc23)CC1]

or

(g) CCT128930, (CAS, 885499-61-6), formula C18H20ClN5, or compound with structure

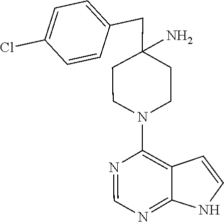 [SMILES: NC1(Cc2ccc(Cl)cc2)CCN(c2ncnc3[nH]ccc23)CC1]

Accordingly the inhibitor of the interaction between AKT and the phospholipids can be Perifosine (D-21266, KRX0401), (CAS 157716-52-4), formula C25H52NO4P, or compound with structure

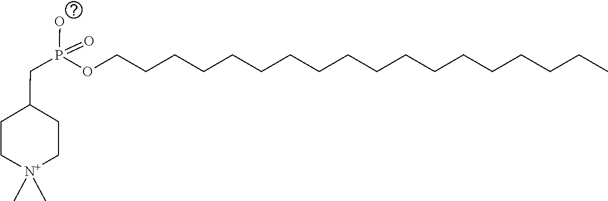 [SMILES: CCCCCCCCCCCCCCCCCCOP(=O)(O)CC1CC[N+](C)(C)CC1]

Preferably the AKT inhibitor is MK-2206 or GSK690693

According to the present invention the AKT inhibitor may be added at a concentration of greater than or equal to between 10 and 1000 fold greater than the IC50 of inhibition of the AKT inhibitor for AKT, optionally IC50 of inhibition of AKT 1, AKT2, or AKT3, preferably at a concentration of greater than or equal to any of 10, 25, 20, 75, 100, 200, 300, 400, 500 600, 700, 800, 900 or 1000 fold of IC50. Preferably the AKT inhibitor may be added at a concentration of between 0.01 uM and 10 uM or 20 uM to 100 uM, preferably at a concentration equal to or greater than 0.01, 0.025, 0.05, 0.075, 0.1, 0.25, 0.5, 0.75, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 uM, preferably 0.5 uM.

T Cells of the Process

According to the present invention the activated and modified and/or transduced T cells or population of T cells produced in the presence of the AKT inhibitor can have an increased or higher relative proportion of any one or more of:

(a) T cells expressing both CD45RA+, CCR7+,

(b) T cells that are CD45RO−, CCR7+, CD45RA+, CD62L+ (L-selectin), CD27+, CD28+ and IL-7Ra+,

(c) T cells that are T_{SCM} cells (stem memory T cells),

(d) T cells with a memory phenotype, preferably CD8 memory phenotype, in comparison to
(i) a population of T cells or a population of activated and modified or transduced T cells, produced in the absence of AKT inhibitor (e.g. reference T cells or population of T cells), or
(ii) a population of T cells or a population of activated and modified or transduced T cells, produced the presence of AKT inhibitor, wherein the AKT inhibitor is added prior to modification or transduction and/or less than or equal to 24 hours after activation or stimulation, for example less than or equal to any one of 1, 2, 3, 4, 5, 6, 7, 8, 9, 10, 11, 12, 13, 14, 15, 16, 17, 18, 19, 20, 21, 22, 23, 24 hours after activation or stimulation (e.g. reference T cells or population of T cells).

According to the present invention the activated and modified and/or transduced T cells or population of T cells produced in the presence of the AKT inhibitor have improved level of any one or more of:

(a) persistence in-vitro and/or in-vivo,

b) expansion from seeding to harvest,

(c) durability of response,

(d) antigen induced cytokine production, optionally interferon gamma production

(e) T cell survival or lifespan or percentage viability,

(f) T cell effector function, preferably cytotoxicity,

preferably in comparison to
(i) a population of T cells or a population of activated and modified or transduced T cells, produced in the absence of AKT inhibitor (e.g. reference T cells or population of T cells), or
(ii) a population of T cells or a population of activated and modified or transduced T cells, produced the presence of AKT inhibitor, wherein the AKT inhibitor is added prior to transduction and/or less than or equal to 24 hours after activation or stimulation, for example less than or equal to any one of 1, 2, 3, 4, 5, 6, 7, 8, 9, 10, 11, 12, 13, 14, 15, 16, 17, 18, 19, 20, 21, 22, 23, 24 hours after activation or stimulation (e.g. reference T cells or population of T cells).

T Cell Persistence

Preferably the activated and modified or transduced T cells or population of T cells demonstrate improved persistence and possesses an increased proportion of less terminally differentiated T cells, preferably within the functional CD8+ population, preferably an increased proportion of double positive SCM cells, preferably expressing both CD45RA+, CCR7+. Preferably the T cells have improved persistence and memory formation, survival, and/or antigen stimulated survival in-vivo and/or when tested in-vitro.

Preferably the activated and modified or transduced T cells or population of T cells demonstrate improved persistence demonstrated in-vivo and/or in-vitro over a time period which is optionally improved relative to reference T cells or population of T cells.

Preferably the activated and modified or transduced T cells or population of T cells demonstrate improved persistence as determined by measurement of increased level of in-vivo expansion and/or increased proportion of double positive SCM cells as quantified, optionally relative to reference T cells or population of T cells, for example by flow cytometry to identify T cells or the proportion of T cells expressing the heterologous TCR or CAR and/or expressing both CD45RA+, CCR7 and/or determined by qPCR to identify the gene-modified T cells.

Preferably the activated and modified or transduced T cells or population of T cells demonstrate improved persistence as determined by improved peak expansion for example as measure of copies/μg by qPCR of heterologous TCR or CAR or median thereof or by measurement of the peak percentage of CD3⁺ cells that are positive for heterologous TC or CAR for example in a sample of in-vivo peripheral blood mononuclear cells (PBMCs) or median thereof. Preferably these values are improved by at least 10%, alternatively 20%, 30%, 40%, 50%, 60%, 70%, 80%, 90%, 100%, 120%, 150%, 200% or more relative to reference T cells or population of T cells. The degree of expansion and the duration of persistence are correlated to tumorigenic response.

Preferably the activated and modified or transduced T cells or population of T cells demonstrate improved persistence as determined by improved levels of cytokine production, for example of interferon gamma production in-vivo and/or in-vitro, as quantified by cytokine assay, for example ELISA or as described herein, optionally relative to reference T cells or population of T cells.

Preferably the activated and modified or transduced T cells or population of T cells demonstrate improved persistence as determined by being less subject to and/or demonstrating a reduction in functional exhaustion in-vitro and/or in-vivo and thereby more able to persist with longer survival time in-vitro and/or in-vivo and provide a longer lasting and more durable immune response, optionally improved relative to reference T cells or population of T cells, for example as determined by assays presented herein. Preferably T-cell function is enhanced by at least 10%, alternatively 20%, 30%, 40%, 50%, 60%, 70%, 80%, 90%, 100%, 120%, 150%, 200% or more compared to reference T cells or population of T cells, for example as judged by increased secretion of y-interferon from CD8+ T-cells, increased T-cell proliferation for example according to cell counting, increased internal signalling for example according to cell signalling assay, increased antigen responsiveness, increased secretion of cytokines and/or interferon, increased target cell killing, increased T-cell activation, increased CD28 signalling, increased T-cell ability to infiltrate tumour, increased ability to recognise and bind to dendritic cell presented antigen.

Preferably the activated and modified or transduced T cells or population of T cells demonstrate improved persistence as determined by having improved potency of antitumor activity, and thereby an improved reduction of tumour immunity or evasion of immune recognition, for example as measured by assay of or determination of degree of tumour infiltration, tumour binding, tumour shrinkage and/or tumour clearance, in-vitro and/or in-vivo, optionally determined compared to reference T cells or population of T cells. Preferably potency of antitumor activity is enhanced or improved by at least 10%, alternatively 20%, 30%, 40%, 50%, 60%, 70%, 80%, 90%, 100%, 120%, 150%, 200% or more relative to reference T cells or population of T cells, for example as measured by tumour binding assay, tumour shrinkage and/or tumour clearance.

Preferably the activated and modified or transduced T cells or population of T cells demonstrate improved persistence as determined by improved or enhanced tumour immunogenicity, for example as measured by the ability to provoke an immune response in response to tumour or tumour antigen, in-vitro and/or in-vivo, for example enhanced by at least 5% or 10%, alternatively 20%, 30%, 40%, 50%, 60%, 70%, 80%, 90%, 100%, 120%, 150%, 200% or more relative to reference T cells or population of T cells, for example as judged by increased secretion of cytokines and/or interferon, increased T-cell proliferation, increased antigen responsiveness for example by in-vitro assay, target cell killing, T-cell activation, CD28 signalling, T-cell ability to infiltrate tumour, ability to recognise and bind to dendritic cell presented antigen.

Preferably the activated and modified or transduced T cells or population of T cells demonstrate improved persistence over a period of time, time period or time course of any one or more of, 5, 6, 7, 8, 9, 10, 11, 12, 13, 14, 15, 16, 17, 18, 19, 20, 21, 22, 23, 24, 25, 26, 27, 28, 29, 30, 31 32, 33, 34, 35, 36 days, and/or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 32, 33, 34, 35, 36, 37, 38, 39, 40, 41, 42, 43, 44, 45, 46, 47, 48, 49 months post infusion in-vivo, or post initiation of culture in-vitro, preferably relative to reference T cells or population of T cells. Preferably the cells demonstrate an improved level of functional activity without exhaustion over the compared time course. Preferably improved persistence is determined over 8 to 14 days post infusion in-vivo.

T Cell Expansion

Preferably the activated and modified or transduced T cells or population of T cells demonstrate improved and/or increased level of T cell expansion as measured in the process or culture between the time points of seeding and harvest. Preferably there is an increased level T cell expansion, division or proliferation in comparison to reference T cells or population of T cells, for example, following T cell activation in comparison to a population of T cells or a population of activated and modified or transduced T cells, produced in the absence of AKT inhibitor (e.g. reference T cells or population of T cells). For example assay or determination of expansion, division or proliferation may be performed using an automated cell counter to provide measurements of cell viability and concentration and rates of proliferation and expansion during the process or culture process or process of the invention or measured in samples therefrom. Preferably the activated and modified or transduced T cells or population of T cells demonstrate improved ability for expansion, division or proliferation in comparison to reference T cells or population of T cells, as measured from equivalent samples or timepoint of samples taken during the process or culture process or process of the invention or measured in samples therefrom, improvement may be determined in assay involving T cell activation. Activation may be initiated in the presence of cytokine, interleukin, antibody, peptide or antigenic peptide as herein before described, for example activation may be through use of a cancer or tumour antigen or peptide thereof, peptide fragment of a cancer or tumour antigen recognised by the heterologous TCR or cell or tissue, for example tumour or cancer cell or tissue presenting the peptide or antigenic peptide, or peptide fragment. Preferably the improvement or comparative improvement with respect to the reference is demonstrated over the period of time or time course as hereinabove described.

T Cell Durability

Accordingly the activated and modified or transduced T cells or population of T cells demonstrate improved durable response and/or durable response rate in-vivo in comparison to reference T cells or population of T cells. Preferably the T cells or population of T cells provide an improved sustained response of reducing tumour growth or tumour growth rate or maintaining tumour size after cessation of treatment/post infusion in-vivo, for example, as determined by the measurement of tumour size or tumour number preferably enhanced by at least 10%, alternatively 20%, 30%, 40%, 50%, 60%, 70%, 80%, 90%, 100%, 120%, 150%, 200% or more relative to such levels before the infusion or treatment or intervention or relative to infusion or treatment with reference T cells or population of T cells. Preferably the improvement or comparative improvement with respect to the reference is demonstrated over the period of time or time course as hereinabove described.

T Cell Cytokine Production

Preferably the activated and modified or transduced T cells or population of T cells demonstrate improved and/or increased level of cytokine production, for example in comparison to reference T cells or population of T cells, in response to a cancer or tumour antigen or peptide thereof, or peptide, antigenic peptide, peptide fragment of a cancer or tumour antigen of or presented by tumour of cancer cell or tissue; and recognised by the heterologous TCR or CAR in comparison to the reference T cells or population of T cells. The cytokine may be Granulocyte-Macrophage Colony-Stimulating Factor (GM-CSF), IFN-γ, IL-2, Tumor Necrosis Factor (TNF)-α, MIP-1β (CCL4), IL-17, IL-10, IL-4, IL-5, IL-13, IL-2 Receptor, IL-12, or MIG (CXCL9); preferably IFNγ, IL-2, TNFα, GM-CSF, or MIP1β; preferably IFNγ, IL-2, TNFα, GM-CSF, and MIP1β. Additionally or alternatively the activated and modified or transduced T cells or population of T cells may demonstrate an induction of an increased level of cytokine production in dendritic cells in response to a cancer or tumour antigen or peptide thereof, or peptide, antigenic peptide, peptide fragment of a cancer or tumour antigen of or presented by tumour of cancer cell or tissue; and recognised by the heterologous TCR or CAR in comparison to the reference T cells or population of T cells. The cytokine may be Granulocyte-Macrophage Colony-Stimulating Factor (GM-CSF), IFN-γ, IL-2, Tumor Necrosis Factor (TNF)-α, MIP-1β (CCL4), IL-17, IL-10, IL-4, IL-5, IL-13, IL-2 Receptor, IL-12, or MIG (CXCL9); preferably IFN-γ, IL-12, or MIG; alternatively IFN-γ, IL-12, and MIG. Suitable assays for determination of cytokine production are known in the art. Preferably the improvement or comparative improvement with respect to the reference is demonstrated over the period of time or time course as hereinabove described.

[symbol]

T Cell Survival, Lifespan, Viability

Preferably the activated and modified or transduced T cells or population of T cells demonstrate an improved and/or increased survival or lifespan or percentage viability in-vitro and/or in-vivo, for example in comparison to reference T cells or population of T cells. Preferably the improvement or comparative improvement with respect to the reference is demonstrated over the period of time or time course as hereinabove described. Preferably the activated and modified or transduced T cells or population of T cells demonstrate improved T cell viability as determined by measuring the level of T cell proliferation in response to antigenic stimulus, for example the antigen specific to the heterologous TCR or CAR, in in-vitro or in-vivo samples for example using dye-based proliferation assay or ³H-thymidine incorporation proliferation assay. Additionally or alternatively T cell viability may be determined by measuring the level of antigen specific response of the T cells in terms of cytokine production of antigen responding T cells for example using enzyme-linked immunosorbent assay (ELISA) or enzyme-linked immunospots (ELISpots). This may be combined with colorimetric assay for physical cellular growth and measurement of marker activity associated with viable T cell number. Preferably the activated and modified or transduced T cells or population of T cells demonstrate improved persistence as determined by measurement of increased survival or lifespan as quantified for example by flow cytometry to identify T cells or the proportion of T cells expressing the heterologous TCR or CAR and/or expressing both CD45RA+, CCR7 and/or determined by qPCR to identify the gene-modified T cells.

T Cell Effector Function

The T cell or population of T cells according to the present invention may demonstrate an improved antigen response or class I antigen response, for example in comparison to reference T cells or population of T cells. Preferably the improvement or comparative improvement with respect to the reference is demonstrated over the period of time or time course as hereinabove described. The T cell or population of T cells according to the invention may demonstrate improved or increased expression of CD40L, affinity for antigen presenting cells and/or, cytokine production, cytotoxic activity for example as determined by cell killing assay of cells expressing T cell recognised antigen, tumour or cancer antigen, induction of dendritic cell maturation or induction of dendritic cell cytokine production, optionally in response to cancer or tumour antigen or peptide or cancer peptide, antigenic peptide, peptide fragment of a cancer or tumour antigen or presented by tumour of cancer cell or tissue and recognised or bound by the T cells or heterologous TCR or CAR.

Compositions and Therapies

The present invention provides T cells or a population of T cells produced according to the method of the present invention.

The present invention further provides a composition comprising T cells or a population of T cells produced according to the method of the present invention and a physiologically acceptable excipient.

A population of T cells or modified T cells according to the invention may be admixed with other reagents, such as buffers, carriers, diluents, preservatives and/or pharmaceutically acceptable excipients. Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990. In some preferred embodiments, the modified T cells, or population of T cells, according to the invention may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The present invention provides T cells or a population of T cells produced according to the method of the present invention or the composition thereof for use in adoptive therapy. Accordingly the modified T cells or population of T cells may be administered, intravenously, intramuscularly, subcutaneously, topically, orally, transdermally, intraperitoneally, intraorbitally, by implantation, by inhalation, intrathecally, intraventricularly, or intranasally or by intravenous infusion. Preferably, the modified immunoresponsive cells may be administered intravenously or by intravenous infusion. Accordingly, the modified T cells or a population of T cells may be administered as a single dose or as more than one dose (multiple doses) and may be administered at a dose of between about 500 million to any one of about 1 billion cells, about 2 billion cells, about 3 billion cells, about 4 billion cells, about 5 billion cells, about 6 billion cells, about 7 billion cells, about 8 billion cells, about 9 billion cells, about 10 billion cells, about 11 billion cells, about 12 billion cells, about 13 billion cells, about 14 billion cells, about 15 billion cells, about 16 billion cells, about 17 billion cells, about 18 billion cells, about 19 billion cells, about 20 billion cells, or about 21 billion cells.

The present invention provides T cells or a population of T cells produced according to the method of the present invention or the composition thereof for use in the treatment of tumour and/or cancer. The tumour and/or cancer may be selected from; lung cancer, non-small cell lung cancer (NSCLC), metastatic or advanced NSCLC, squamous NSCLC, adenocarcinoma NSCLC, adenosquamous NSCLC, large cell NSCLC, ovarian cancer, gastric cancer, urothelial cancer, esophageal cancer, esophagogastric junction cancer (EGJ), melanoma, bladder cancer, head and neck cancer, head and neck squamous cell carcinoma (HNSCC), cancer of the oral cavity, cancer of the oropharynx, cancer of the hypopharynx, cancer of the throat, cancer of the larynx, cancer of the the tonsil, cancer of the tongue, cancer of the soft palate, cancer of the pharynx, synovial sarcoma, myxoid round cell liposarcoma (MRCLS). According to the present invention the cancer may be selected from any one of breast cancer, metastatic breast cancer, liver cancer, renal cell carcinoma, synovial sarcoma, urothelial cancer or tumour, pancreatic cancer, colorectal cancer, metastatic stomach cancer, metastatic gastric cancer, metastatic liver cancer, metastatic ovarian cancer, metastatic pancreatic cancer, metastatic colorectal cancer, metastatic lung cancer, colorectal carcinoma or adenocarcinoma, lung carcinoma or adenocarcinoma, pancreatic carcinoma or adenocarcinoma, mucinous adenoma, ductal carcinoma of the pancreas, hematological malignancy. Optionally the cancer and/or tumour may express MAGE-A4 or AFP or an antigen peptide or a peptide antigen of MAGE A4 and/or comprises the amino acid sequence GVYDGREHTV, SEQ ID NO: 1 (MAGE A4) or an antigen peptide or a peptide antigen of alpha fetoprotein (AFP) and/or comprises the sequence FMNKFIYEI (SEQ ID No: 2) or residues 158-166 derived from alpha fetoprotein (AFP) (SEQ ID NO: 3).

Treatment

Preferably the treatment extends or improves or effectively extends or effectively improves:

(a) progression free survival,

(b) time to progression,

(c) duration of response,

(d) overall survival,

(e) objective response or objective response rate,

(f) overall response or overall response rate,

(g) partial response or partial response rate,

(h) complete response or complete response rate;

(i) stable disease rate or median stable disease

(j) median progression free survival,

(k) median time to progression,

(l) median duration of response, or

(m) median overall survival;

(n) median objective response or median objective response rate,

(o) median overall response or median overall response rate,

(p) median partial response or median partial response rate,

(q) median complete response or median complete response,

(r) median stable disease rate or median stable disease,

in comparison to treatment comprising reference T cells or population of T cells are herein above described.

“Overall survival” refers to a subject remaining alive for a defined period of time. “Objective response rate” (ObRR) is the proportion of subjects with tumour size reduction of a predefined amount, optionally determined by sum of the longest diameters (SLD) of target lesions or tumours, and for a minimum time period. “Overall response rate (ORR)” is defined as the proportion of subjects who have a partial or complete response to therapy; it does not include stable disease. ORR is generally defined as the sum of complete responses (CR) and partial responses (PRs) over a specified time period. “Progression free survival” (PFS) refers to the time from treatment (or randomization) to first disease progression or death. “Time to progression” (TTP) does not count patients who die from causes other than the cancer or tumour being treated but is otherwise equivalent to PFS. “Duration of response” (DoR), is the length of time that cancer, tumour or lesion continues to respond to treatment without growing or spreading. According to the invention DoR, TTP and PFS can be assessed by Response Evaluation Criteria in Solid Tumors (RECIST) or can be assessed by CA-125 levels as a determinant of progression.

According to the invention a “complete response” (CR) is determined where all target lesions or tumours have been assessed or measured as having disappeared. “Partial response” (PR) is determined when there is a measurement of an at least a 30% decrease in the sum of the longest diameters (SLD) of target lesions or tumours, for example as referenced to the control or pre-treatment comparator. “Progressive disease” (PD) is determined when there is a measurement of at least a 20% increase in the sum of the longest diameters (SLD) of target lesions or tumours, for example as referenced to the control or pre-treatment comparator, since the treatment started or the presence of one or more new lesions. “Stable disease” (SD) is determined where it is determined that there is neither sufficient reduction or decrease in the sum of the longest diameters (SLD) of target lesions or tumours to qualify for PR, nor sufficient increase to qualify for PD, taking as reference the smallest SLD since the treatment started.

According to the invention there is provided;

(a) a kit comprising comprising an effective amount of T cells or a population of T cells produced according to the method of the present invention and a package insert comprising instructions for using the T cells to treat or delay the progression of cancer and/or tumour in a subject.

The invention will be further described by reference to the following figures and examples.

### FIGURES

FIG. 1. Timepoints for addition of AKTi. Schematic diagram showing the timepoints at which MK-2206 was added to T cell expansion culture, comparison was made between cultures with a day 0+day 2 addition vs day 2 alone. Day 0 addition occurs post bead addition, T-Cell activation, but pre-transduction/expansion. Day 2 addition occurs post transduction but pre-expansion. Added to media used in G-Rex top up, does not disrupt process.

FIG. 2. T Cell Total Nucleated Cell (TNC) counts in Response to MK-2206 for healthy donor materials (HDM) HDM were expanded in 10 M G-Rex for 10 days. Cultures were treated with MK-2206 at various concentrations added at 3 different timepoints; day 0(+D2), Day 2. The total nucleated cell (TNC) counts were taken on day 10 post-harvest on the auto cell counter ViCELL.

FIG. 3 Multiple Healthy Donor expansion on day 10 harvest following MK-2206 compound addition. Six healthy donor materials were expanded for 10 days in 10 M G-Rex following the described process. Cultures were treated with MK-2206 at concentrations 10 uM titrated down to 0.05 uM. Graph A detail compounds added on day 0 (+day 2 media addition), Graph B details compounds added on day 2 alone. Harvested cells were counted using the ViCELL. Results displayed as fold change in expansion to untreated control. Where replicate cultures were expanded, an average of 2 replicates was shown for datapoint+/−SEM.

FIG. 4. Healthy Donor Memory Phenotype Distribution in transduced CD8+ compartments. Healthy donor material was expanded for 10 days in 10 M G-Rex following the described process. Cultures were treated with AKTi MK-2206 at multiple concentrations in uM shown on X-axis. The compounds were added either on day 0+day 2 or day 2 alone. Harvested cells were analysed by flow cytometry gated CD3+/Dextramer+/CD8+ compartment for Memory Phenotype Markers (CCR7+/−, CD45RA+/−).

FIG. 5. Antigen-stimulated IFNgamma secretion by T-cells expanded in the presence of MK-2206 Expanded T-Cells were thawed from frozen, rested for 2 hours, and then co-cultured with Antigen-positive cell line (A375, seeded a day before) for 48 hours. Supernatants from stimulated T-Cell cultures were analysed for cytokine release using IFNγ ELISA. Each datapoint shows an average of 3 replicates+/−SEM. MK-2206 was added on day 0 +day 2, Day 2.

### EXAMPLES

### Example 1

1.1. Objectives

The objective of the following study was to investigate new processes of producing T cells in culture which could lead to an improved T cell function without detriment to T cell expansion, such that the T cell population produced would have improved functional effectiveness in adoptive therapy and in the treatment of cancer. It was particularly desirable for the process to increase the proportion of the less differentiated T cell population within functional CD8+ compartment, particularly with minimal or no negative impact on T cell expansion, T cell viability, transduction, CD8 frequency in final population and also having an improved cytokine secretion in response to antigen specific activation. It was expected that such a cell population would have improved anti-tumour activity as gauged by effector function in response to antigen (cytotoxic cell killing activity and cytokine production), and demonstrate improved persistence as gauged by prolonged survival in response to antigen stimulated survival, additionally the T cells would have improved persistence and memory formation, survival, and antigen stimulated survival. The process incorporates the use of an AKT inhibitor, MK-2206 is exampled here which is a small molecule inhibitor of Protein Kinase B (AKT), which acts upstream of the glycolysis pathway. Improved T cell expansion was achieved when MK-2206 was added on Day 2 after activation of the T cells on day 0 and transduction on day 1, analysis showed improved functionality of the resultant T cell population as explained below.

1.2. T Cell Isolation Expansion and Culture

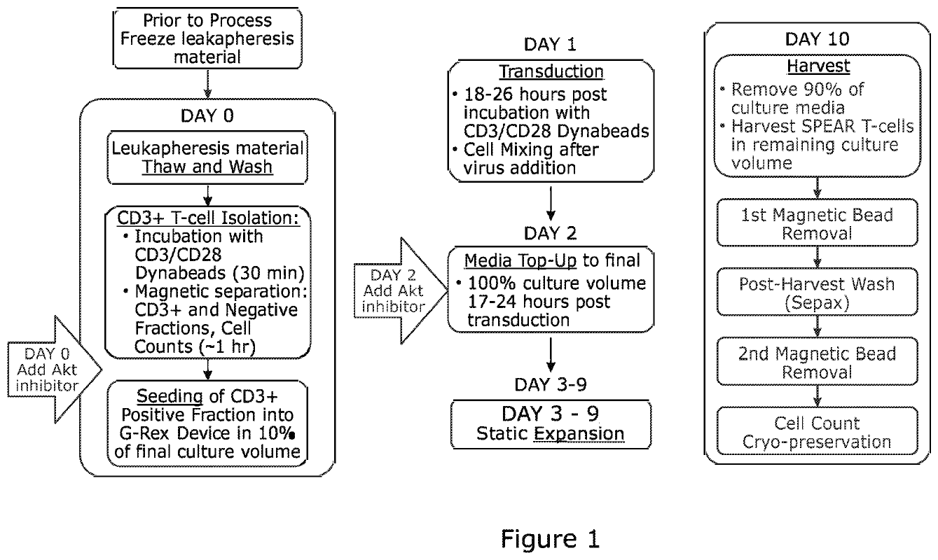

All cells were expanded and transduced, using a static expansion system, (scalable G-Rex®). Cryopreserved leukapheresis starting materials from healthy donors or cancer patients were thawed, washed, activated with CD3/CD28 magnetic Dynabeads, and CD3+ positive fraction was magnetically separated. The starting leukapheresis material was similar across the healthy donors. CD3+ cells ranged from 44-64%, all with an increased purity of 79-87% following positive isolation using anti CD3/CD28 Dynabeads. CD3+ phenotyping identified a range of stem cell memory (SCM) markers (CCR7+/CD45RA+) ranging from 11.6-57% in the CD8+ compartment between individuals. Enriched CD3+ T cells were seeded based on total nucleated cell (TNC) counts at 1.5×10⁶ TNC/cm² density in the G-Rex® static cell expansion system device, in 10% of final culture volume using TexMACS+5% human AB serum (HABS) supplemented with 100 IU/ml IL-2. Transduction with vector expressing a heterologous TCR (recognising MAGE-A4) was performed 18-26 hours post addition of CD3/CD28 Dynabeads to the cells, using lentivirus (LV) vector at a MOI of 0.45. Media was then added to top up to the final culture volume, this was performed 17-24 hours post transduction with TexMACS+5% HABS+500 IU/ml IL-2. Cells were then incubated at 37° C., 5% CO₂ for a further 8 days. MK-2206 was added to the media at defined intervals; in day 0 seeding media and supplemented in media used for day 2 media addition, or solely supplemented in media for day 2 media addition. The concentration of the compound varied for each experiment, 10 uM, 7.5 uM, 5 uM, 2.5 uM, 2 uM, 1.5 uM, 1 uM, 0.5 uM, 0.25 uM, 0.1 uM, 0.05 uM as dictated in the following examples. T cells were harvested 10 days post seeding. After bead removal, T-cells were counted by automated cell analysis (with ViCELL). Harvested cells were then frozen for later phenotypic and functional analysis as described. An overview of the process is given in FIG. 1.

1.3 Antigen-Stimulation and Cytokine Release Analysis (IFNγ)

Function of expanded T-cells was evaluated based on the levels of cytokine release by expanded T-cells following antigen-stimulation with the MAGEA4-expressing cell line, A375. A375 MAGEA4 positive target cells were counted using automated cell counter and seeded in a 96 well U-bottom plate at 30,000 viable nucleated cells (VNC) per well, in 100 uL volume per well, one day prior to T-cell stimulation and incubated overnight at 37° C., 5% CO₂. Wells that were used as “T cells alone” control, received 100 uL R10 media (RPMI medium with 10% FBS and 1% Penicillin/Streptomycin) only. Following overnight incubation of target cells, harvested and frozen T-cells were thawed at 37° C. in the water bath and washed with R10 media. Cells were rested in R10 media at a density of 2×10⁶ VNC/mL for 2 hours at 37° C., 5% CO₂. After 2-hour rest, T-cells were counted using automated cell counter, and cultures were normalised to 35% transduction efficiency for all donors combining calculated amount of transduced and non-transduced VNC for each condition. The normalised T-cells were seeded at 150,000 VNC/well in 100 uL volume to the plate containing A375 target cells, seeded a day before. Plates were returned to the incubator at 37° C., 5% CO₂ for 48 hours to allow for T-cell stimulation. Following 48-hour stimulation, plates were centrifuged for 5 minutes at 400 G and supernatants transferred to a new 96 well plate. Supernatants were stored at −20° C.

ELISAs were performed using Human IFNγ by ELISA. Standard controls were prepared for the plate to a maximum concentration of 10000 pg/ml for IFNy and serially diluted 1 in 2 to generate a standard curve for each assay. Colorimetric read-out was analysed (BMG LABTECH FLUOstar Omega plate reader) at OD 450.

### Example 2

2.1. T Cell Expansion Analysis

Healthy donor material (leukapheresis material comprising white blood cells separated from a donor sample of blood) was processed for culture in static expansion (G-Rex device) following the aforementioned process for a 10 day duration. The cells were cultured with MK-2206 (1.25, 2.5, 5.0, 10 uM) added to cultures on day 0+day 2 or day 2 alone (with the day 2 media addition). On harvest, Total Nucleated Cell (TNC) counts were performed using an automated cell counter (ViCELL) to provide expansion data, and flow cytometry performed to determine phenotype. The harvested cultures were frozen and later thawed for functional assessment, an antigen stimulated cytokine release assay (1.3 described above).

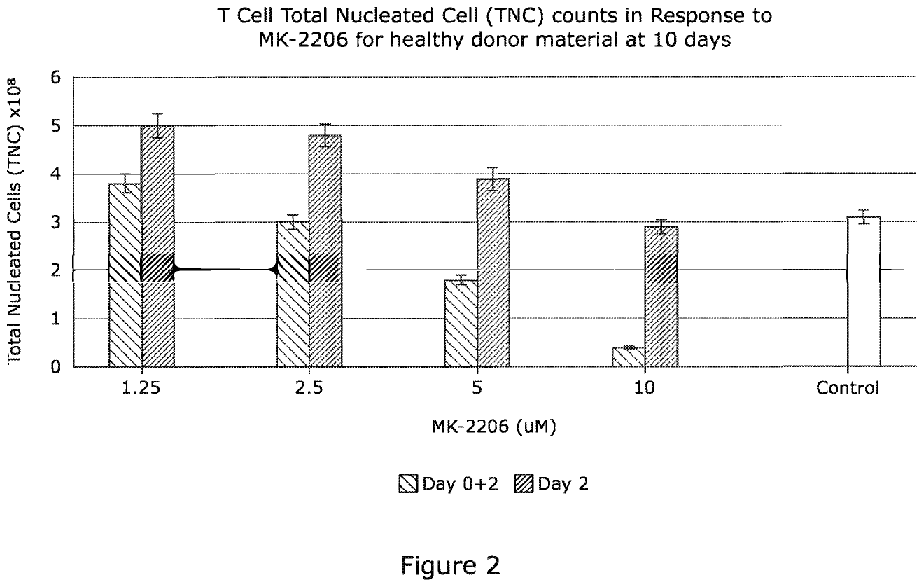

The data (FIG. 2) showed increased expansion in the presence of the lower concentrations of MK-2206 when compared to the untreated control. All timepoints for addition of MK-2206 showed reduced expansion at 10 uM. With exception of 10 uM, day 2 alone addition provided improved TNC yield. Day 0(+2) was consistently providing low yield.

### Example 3

3.1. Extended Titration and Expansion Analysis

The concentration of MK-2206 was tested in further titratration from 0.05 to 10 uM to fully determine optimal dosing and day 0(+2) and day 2 alone are both assessed to determine the optimal time of addition. Again, expansion, phenotype and cytokine production data has been assessed. An additional four donors were tested at the varying concentrations, to provide a full titration range in this study, 10 uM to 0.05 uM. All donors were tested in 10 M G-Rex following the described process and were harvested on Day 10. Expansion data considered the timepoint of addition of MK-2206; Day 0+2 and Day 2 alone.

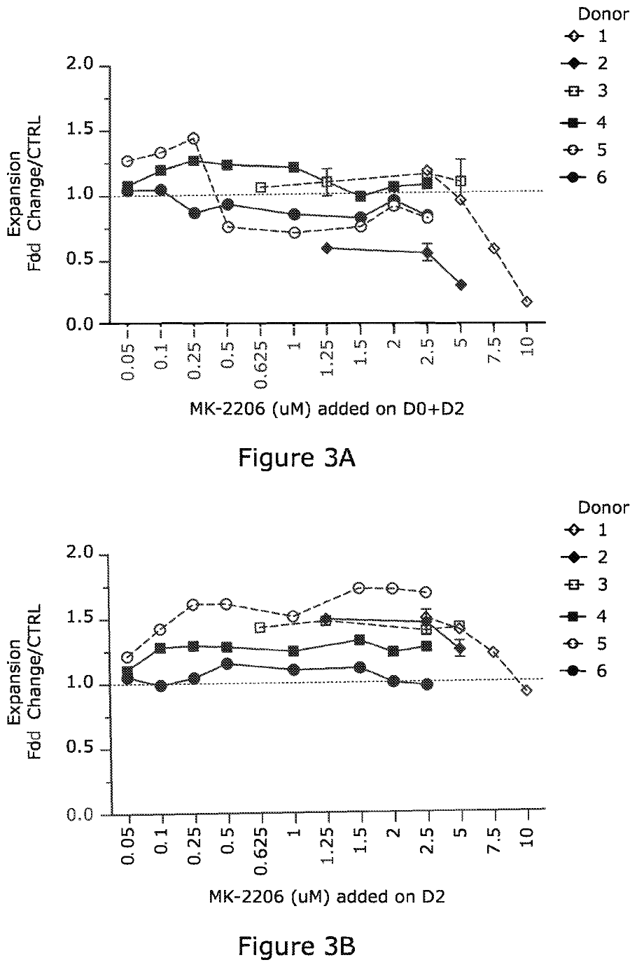

The addition timepoint of MK-2206 has an impact on expansion. Day 0+2 did not show a consistent response. There was donor variation across all concentrations tested, with limits of expansion increase of 1.5-fold and expansion decrease of 0.15 fold. Peak expansion was at the lowest concentrations (0.05 and 0.1 uM), where all donors responded similar or 0.5 fold better than the untreated control (FIG. 3A). Day 2 addition had no negative impact on expansion. Donor variation did exist; however, all conditions provided a similar or better response to the control (≥1-2fold). Day 2 addition provided peak expansion for 3 donors at 0.5 uM, before expansion began to decrease in a dose dependent response (0.05-0.25 uM). Expansion response appears to plateau >0.5 uM, with some donor variation (FIG. 3B). Overall, Day 2 addition provided a measurably better response to expansion that of Day 0+2.

### Example 4

3.4. T Cell Phenotype Analysis

Flow cytometry was performed on healthy donor material on day 10 harvest from the described process to determine Memory Phenotype markers of cultured cells using CCR7 and CD45RA staining

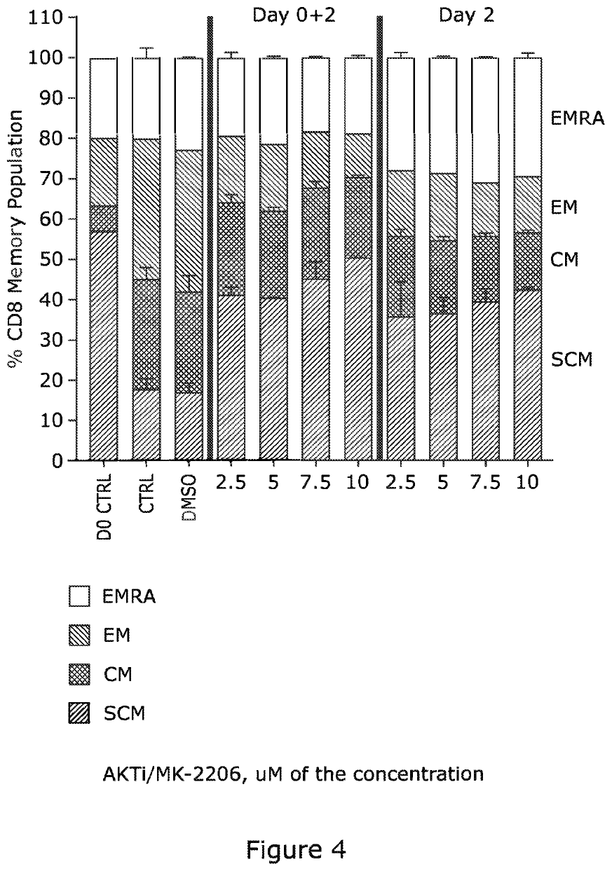

FIG. 4 data shows that cells cultured with MK-2206 showed increased CCR7+CD45RA+ populations at harvest when added on day 0+2 and day 2 alone when compared to the untreated control. The concentration of additive did not have a significant effect on increasing CCR7+CD45RA+. The increase in these stem cell like markers were more reflective of the day 0 starting leukapheresis material (FIG. 4 D0), suggesting perhaps that the additive maintains this population by slowing differentiation.

MK-2206 has an impact on memory phenotype markers (CCR7/CD45RA), as shown (FIG. 4), there is also an observed increase in CD62L expression, increasing the population of these double positive SCM-like cells which are less terminally differentiated less subject to functional exhaustion and more able to persist with longer survival time in-vivo and provide a longer lasting and more durable immune response. This increase in CCR7+ populations indicates an increase in Stem Cell-like and central Memory markers, and a consequent decrease in CCR7−CD45RA−, i.e. the more terminally differentiated effector memory (EM) populations, this cell subpopulation has potential for exhaustion and decreased T-Cell survival and proliferation in-vivo. The EMRA population generally remains similar to the untreated control across all concentrations tested. The increase in CCR7+ population signifies that cells cultures with MK-2206 are generating ‘fitter’ T-Cells which have the ability to survive and persist, this is of functional benefit for the T- Cell population particularly in the context of adoptive therapy and treatment of cancer.

SCM T cells are “stem memory cells” (T_{SCM} cells) CCR7+/CD45RA+ and have similarity to naive T cells, they also express large amounts of CD95, IL-2R beta, CXCR3, and LFA-1, and show numerous functional attributes distinctive of memory cells. SCM T cells are less terminally differentiated T cells with long term response and high capability for self renewal and survival. CM T cells are “central memory T cells” (T_{CM} cells) express CCR7+/CD45RA− and also have intermediate to high expression of CD44. CM T cells are a memory subpopulation commonly found in the lymph nodes and in the peripheral circulation. EM T cells are “effector memory T cells” (T_{EM} cells) which are CCR7-/CD45RA− i.e. but lack expression of CCR7 and CD45RA. They also have intermediate to high expression of CD44. These memory T cells lack lymph node-homing receptors and are thus found in the peripheral circulation and tissues, they are immediate response effector cells and subject to exhaustion of function due to their terminally differentiated state. EMRA cells (T_{EMRA}) are terminally differentiated effector memory cells re-expressing CD45RA (CCR7−/CD45RA+), which is a marker usually found on naive T cells.

### Example 5

5.1. Cytokine Production

Expanded T-cells derived from the T cell culture process described above were thawed and seeded in the presence of MAGEA4-positive cell line, A375. For the cytokine release assays, the target and effector cells were co-cultured for 48 hours then the supernatants were collected and analysed for IFN gamma levels by ELISA. Non-transduced controls were also included in the assay as an additional control measure. All samples were normalised to a 35% transduction rate.

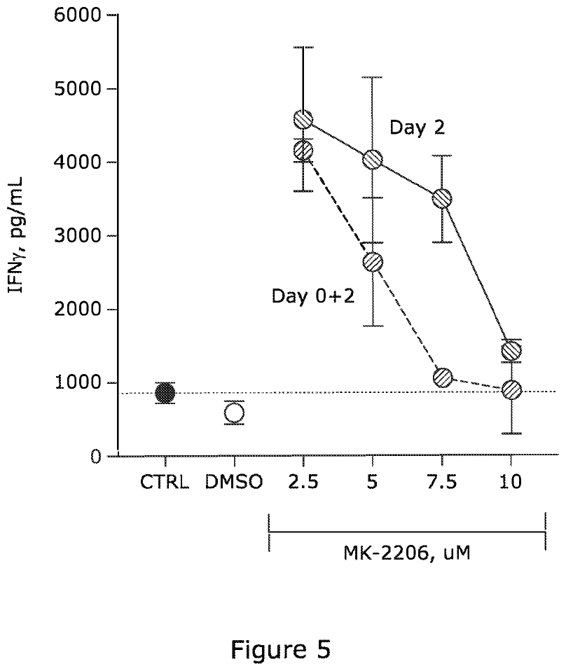

The levels of IFN gamma produced by antigen stimulated T-Cells was investigated as an indication of T cell population functionality. MK-2206 was assessed against the untreated control for each donor. The response in IFN gamma production following antigen stimulation was to a degree donor dependent. The increase in MK-2206 concentration also appeared to decrease the cytokine production of the cells (>5 uM). The highest increase was seen with day 2 addition of MK-2206 (FIG. 5).

### Sequences

GVYDGREHTV, (SEQ ID NO: 1), MAGE A4 peptide
FMNKFIYEI (SEQ ID No: 2) alpha fetoprotein (AFP) peptide or residues 158-166 derived from SEQ
ID NO: 3
Human Alpha-fetoprotein SEQ ID NO: 3
MKWVESIFLI FLLNFTESRT LHRNEYGIAS ILDSYQCTAE ISLADLATIF
FAQFVQEATY KEVSKMVKDA LTAIEKPTGD EQSSGCLENQ LPAFLEELCH
EKEILEKYGH SDCCSQSEEG RHNCFLAHKK PTPASIPLFQ VPEPVTSCEA
YEEDRETFMN KFIYEIARRH PFLYAPTILL WAARYDKIIP SCCKAENAVE
CFQTKAATVT KELRESSLLN QHACAVMKNF GTRTFQAITV TKLSQKFTKV
NFTEIQKLVL DVAHVHEHCC RGDVLDCLQD GEKIMSYICS QQDTLSNKIT
ECCKLTTLER GQCIIHAEND EKPEGLSPNL NRFLGDRDFN QFSSGEKNIF
LASFVHEYSR RHPQLAVSVI LRVAKGYQEL LEKCFQTENP LECQDKGEEE
LQKYIQESQA LAKRSCGLFQ KLGEYYLQNA FLVAYTKKAP QLTSSELMAI
TRKMAATAAT CCQLSEDKLL ACGEGAADII IGHLCIRHEM TPVNPGVGQC
CTSSYANRRP CFSSLVVDET YVPPAFSDDK FIFHKDLCQA QGVALQTMKQ
EFLINLVKQK PQITEEQLEA VIADFSGLLE KCCQGQEQEV CFAEEGQKLI
SKTRAALGV
SEQ ID NO: 4; MAGE A4 TCR α chain, CDRs bold underlined
MKKHLTTFLVILWLYFYRGNGKNQVEQSPQSLIILEGKNCTLQCNYTVSPFSNLRWYKQDTG
RGPVSLTILTFSENTKSNGRYTATLDADTKQSSLHITASQLSDSASYICVVSGGTDSWGKLQ
FGAGTQVVVTPDIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNFQN
LSVIGFRILLLKVAGFNLLMTLRLWSSGSRAKR
SEQ ID NO: 5; MAGE A4 TCR α chain coding sequence
ATGAAGAAGCACCTGACCACCTTTCTCGTGATCCTGTGGCTGTACTTCTACCGGGGCAAC
GGCAAGAACCAGGTGGAACAGAGCCCCCAGAGCCTGATCATCCTGGAAGGCAAGAACT
GCACCCTGCAGTGCAACTACACCGTGTCCCCCTTCAGCAACCTGCGGTGGTACAAGCAGG
ACACCGGCAGAGGCCCTGTGTCCCTGACCATCCTGACCTTCAGCGAGAACACCAAGAGC
AACGGCCGGTACACCGCCACCCTGGACGCCGATACAAAGCAGAGCAGCCTGCACATCAC
CGCCAGCCAGCTGAGCGATAGCGCCAGCTACATCTGCGTGGTGTCCGGCGGCACAGACA
GCTGGGGCAAGCTGCAGTTTGGCGCCGGAACACAGGTGGTCGTGACCCCCGACATCCAG
AACCCTGACCCTGCCGTGTACCAGCTGCGGGACAGCAAGAGCAGCGACAAGAGCGTGTG
CCTGTTCACCGACTTCGACAGCCAGACCAACGTGTCCCAGAGCAAGGACAGCGACGTGT
ACATCACCGACAAGACCGTGCTGGACATGCGGAGCATGGACTTCAAGAGCAATAGCGCC
GTGGCCTGGTCCAACAAGAGCGACTTCGCCTGCGCCAACGCCTTCAACAACAGCATTATC
CCCGAGGACACATTCTTCCCAAGCCCCGAGAGCAGCTGCGACGTCAAGCTGGTGGAAAA
GAGCTTCGAGACAGACACCAACCTGAACTTCCAGAACCTGAGCGTGATCGGCTTCAGAA
TCCTGCTGCTGAAGGTGGCCGGCTTCAACCTGCTGATGACCCTGAGACTGTGGTCCAGCG
GCAGCCGGGCCAAGAGA
SEQ ID NO: 6; (MAGE A4 TCR β chain) CDRs bold underlined
MASLLFFCGAFYLLGTGSMDADVTQTPRNRITKTGKRIMLECSQTKGHDRMYWYRQDPGL
GLRLIYYSFDVKDINKGEISDGYSVSRQAQAKFSLSLESAIPNQTALYFCATSGQGAYEEQFF
GPGTRLTVLEDLKNVFPPEVAVFEPSEAEISHTQKATLVCLATGFYPDHVELSWWVNGKEVH
SGVSTDPQPLKEQPALNDSRYCLSSRLRVSATFWQNPRNHFRCQVQFYGLSENDEWTQDRA
KPVTQIVSAEAWGRADCGFTSESYQQGVLSATILYEILLGKATLYAVLVSALVLMAMVKRK
DSRG
SEQ ID NO: 7; (MAGE A4 TCR β chain coding sequence)
ATGGCCAGCCTGCTGTTCTTCTGCGGCGCCTTCTACCTGCTGGGCACCGGCTCTATGGAT
GCCGACGTGACCCAGACCCCCCGGAACAGAATCACCAAGACCGGCAAGCGGATCATGCT
GGAATGCTCCCAGACCAAGGGCCACGACCGGATGTACTGGTACAGACAGGACCCTGGCC
TGGGCCTGCGGCTGATCTACTACAGCTTCGACGTGAAGGACATCAACAAGGGCGAGATC
AGCGACGGCTACAGCGTGTCCAGACAGGCTCAGGCCAAGTTCAGCCTGTCCCTGGAAAG
CGCCATCCCCAACCAGACCGCCCTGTACTTTTGTGCCACAAGCGGCCAGGGCGCCTACGA
GGAGCAGTTCTTTGGCCCTGGCACCCGGCTGACAGTGCTGGAAGATCTGAAGAACGTGTT
CCCCCCAGAGGTGGCCGTGTTCGAGCCTTCTGAGGCCGAAATCAGCCACACCCAGAAAG
CCACACTCGTGTGTCTGGCCACCGGCTTCTACCCCGACCACGTGGAACTGTCTTGGTGGG
TCAACGGCAAAGAGGTGCACAGCGGCGTGTCCACCGATCCCCAGCCTCTGAAAGAACAG
CCCGCCCTGAACGACAGCCGGTACTGCCTGAGCAGCAGACTGAGAGTGTCCGCCACCTT
CTGGCAGAACCCCAGAAACCACTTCAGATGCCAGGTGCAGTTTTACGGCCTGAGCGAGA
ACGACGAGTGGACCCAGGACAGAGCCAAGCCCGTGACACAGATCGTGTCTGCCGAAGCT
TGGGGGCGCGCCGATTGTGGCTTTACCAGCGAGAGCTACCAGCAGGGCGTGCTGAGCGC
CACCATCCTGTACGAGATCCTGCTGGGAAAGGCCACACTGTACGCCGTGCTGGTGTCTGC
CCTGGTGCTGATGGCCATGGTCAAGCGGAAGGACAGCCGGGGC
SEQ ID NO: 8; (MAGE A4 TCR α chain variable region)136AA - CDRs bold underlined
MKKHLTTFLVILWLYFYRGNGKNQVEQSPQSLIILEGKNCTLQCNYTVSPFSNLRWYKQDTG
RGPVSLTILTFSENTKSNGRYTATLDADTKQSSLHITASQLSDSASYICVVSGGTDSWGKLQ
FGAGTQVVVTPD
SEQ ID NO: 9; (MAGE A4 TCR β chain variable region)133AA - CDRs bold underlined
MASLLFFCGAFYLLGTGSMDADVTQTPRNRITKTGKRIMLECSQTKGHDRMYWYRQDPGL
GLRLIYYSFDVKDINKGEISDGYSVSRQAQAKFSLSLESAIPNQTALYFCATSGQGAYEEQFF
GPGTRLTVLE
SEQ ID NO: 10; CDRI MAGE A4 TCR α chain
VSPFSN
SEQ ID NO: 11; CDR2 MAGE A4 TCR α chain
LTFSEN
SEQ ID NO: 12; CDR3 MAGE A4 TCR α chain
CVVSGGTDSWGKLQF
SEQ ID NO: 13; CDRI MAGE A4 TCR β chain
KGHDR
SEQ ID NO: 14; CDR2 MAGE A4 TCR β chain
SFDVKD
SEQ ID NO: 15; CDR3 MAGE A4 TCR β chain,
CATSGQGAYEEQFF
Parental AFP TCR TRAV12-2*02/TRAJ41*01/TRAC alpha chain amino acid extracellular sequence
(SEQ ID No: 16)
Q K E V E Q N S G P L S V P E G A I A S L N C T Y S D
R G S Q S F F W Y R Q Y S G K S P E L I M S I Y S N G
D K E D G R F T A Q L N K A S Q Y V S L L I R D S Q P
S D S A T Y L C A V N S D S G Y A L N F G K G T S L L
V T P H I Q N P D P A V Y Q L R D S K S S D K S V C L
F T D F D S Q T N V S Q S K D S D V Y I T D K T V L D
M R S M D F K S N S A V A W S N K S D F A C A N A F N
N S I I P E D T F F P S P E S S
Parental AFP TCR alpha chain DNA sequence (SEQ ID No: 17)
caaaaagaagttgagcagaattctggacccctcagtgttccagagggagccattgcctctctcaactgcacttacagtgaccgaggttcccagtcctt
cttctggtacagacaatattctgggaaaagccctgagttgataatgtccatatactccaatggtgacaaagaagatggaaggtttacagcacagctca
ataaagccagccagtatgtttctctgctcatcagagactcccagcccagtgattcagccacctacctctgtgccgtgaatagtgattccgggtatgcac
tcaacttcggcaaaggcacctcgctgttggtcacaccccatatccagaaccctgaccctgccgtgtaccagctgagagactctaagtcgagtgaca
agtctgtctgcctattcaccgattttgattctcaaacaaatgtgtcacaaagtaaggattctgatgtgtatatcacagacaaatgtgtgctagacatgagg
tctatggacttcaagagcaacagtgctgtggcctggagcaacaaatctgactttgcatgtgcaaacgccttcaacaacagcattattccagaagacac
cttcttccccagcccagaaagttcc
Parental AFP TCR TRBV9*01/TRBD2/TRBJ2-7*01/TRBC2 beta chain amino acid extracellular
sequence (SEQ ID No: 18)
D S G V T Q T P K H L I T A T G Q R V T L R C S P R S
G D L S V Y W Y Q Q S L D Q G L Q F L I Q Y Y N G E E
R A K G N I L E R F S A Q Q F P D L H S E L N L S S L
E L G D S A L Y F C A S S L G G E S E Q Y F G P G T R
L T V T E D L K N V F P P E V A V F E P S E A E I S H
T Q K A T L V C L A T G F Y P D H V E L S W W V N G K
E V H S G V S T D P Q P L K E Q P A L N D S R Y C L S
S R L R V S A T F W Q N P R N H F R C Q V Q F Y G L S
E N D E W T Q D R A K P V T Q I V S A E A W G R A D
Parental AFP TCR beta chain DNA sequence (SEQ ID No: 19)
gattctggagtcacacaaaccccaaagcacctgatcacagcaactggacagcgagtgacgctgagatgctcccctaggtctggagacctctctgtg
tactggtaccaacagagcctggaccagggcctccagttcctcattcagtattataatggagaagagagagcaaaaggaaacattcttgaacgattct
ccgcacaacagttccctgacttgcactctgaactaaacctgagctctctggagctgggggactcagctttgtatttctgtgccagcagcctcggggg
ggaatctgagcagtacttcgggccgggcaccaggctcacggtcacagaggacctgaaaaacgtgttcccacccgaggtcgctgtgtttgagccat
cagaagcagagatctcccacacccaaaaggccacactggtgtgcctggccaccggtttctaccccgaccacgtggagctgagctggtgggtgaat
gggaaggaggtgcacagtggggtctgcacagacccgcagcccctcaaggagcagcccgccctcaatgactccagatacgctctgagcagccg
cctgagggtctcggccaccttctggcaggacccccgcaaccacttccgctgtcaagtccagttctacgggctctcggagaatgacgagtggaccc
aggatagggccaaacccgtcacccagatcgtcagcgccgaggcctggggtagagcagac
Variant AFP TCR (AFP TRAV12-2*02/TRAJ41*01/TRAC alpha chain amino acid extracellular
sequence (SEQ ID No: 20)
QEVEQNSGPLSVPEGAIASLNCTYSDRGSQAFFWYRQYSGKSPELIMSIYSNGDKEDGRFT
AQLNKASQYVSLLIRDSQPSDSATYLCAVNSQSGYALNFGKGTSLLVTPHIQNPDPAVYQLR
DSKSSDKSVCLFTDFDSQTNVSQSKDSDVYITDKTVLDMRSMDFKSNSAVAWSNKSDFACA
NAFNNSIIPEDTFFPSPESS
Variant AFP TCR TRBV9*01/TRBD2/TRBJ2-7*01/TRBC2 beta chain amino acid extracellular
sequence (SEQ ID No: 21)
DSGVTQTPKHLITATGQRVTLRCSPRSGDLSVYWYQQSLDQGLQFLIQYYNGEERAKGNILE
RFSAQQFPDLHSELNLSSLELGDSALYFCASSLGGESEQYFGPGTRLTVTEDLKNVFPPEVAV
FEPSEAEISHTQKATLVCLATGFYPDHVELSWWVNGKEVHSGVSTDPQPLKEQPALNDSRYC
LSSRLRVSATFWQNPRNHFRCQVQFYGLSENDEWTQDRAKPVTQIVSAEAWGRAD
DRGSQS (αCDR1), AFP TCR, SEQ ID NO: 22
IYSNGD (αCDR2), AFP TCR, SEQ ID NO: 23
AVNSDSGYALNF (αCDR3), AFP TCR, SEQ ID NO: 24
SGDLS (βCDR1), AFP TCR, SEQ ID NO: 25
YYNGEE (βCDR2), AFP TCR, SEQ ID NO: 26
ASSLGGESEQY (PCDR3), AFP TCR, SEQ ID NO: 27
DRGSQA (αCDR1), AFP TCR, SEQ ID NO: 28
AVNSDSSYALNF (αCDR2), AFP TCR, SEQ ID NO: 29
AVNSDSGVALNF (αCDR2), AFP TCR, SEQ ID NO: 30
AVNSQSGYALNF (αCDR2), AFP TCR, SEQ ID NO: 31
AVNSQSGYSLNF (αCDR2), AFP TCR, SEQ ID NO: 32
AVNSQNGYALNF (αCDR2), AFP TCR, SEQ ID NO: 33
DRGSFS (αCDR1), AFP TCR, SEQ ID NO: 34
DRGSYS (αCDR1), AFP TCR, SEQ ID NO: 35
AVNSQSSYALNF (αCDR2), AFP TCR, SEQ ID NO: 36
MALPVTALLLPLALLLHAARPSQFRVSPLDRTWNLGETVELKCQVLLSNPTSGCSWLFQPRGA
AASPTFLLYLSQNKPKAAEGLDTQRFSGKRLGDTFVLTLSDFRRENEGYYFCSALSNSIMYF
SHFVPVFLPAKPTTTPAPRPPTPAPTIASQPLSLRPEACRPAAGGAVHTRGLDFACDIYIWAPL
AGTCGVLLLSLVITLYCNHRNRRRVCKCPRPVVKSGDKPSLSARYV, (CD8α), CDRs bold
underlined, signal sequence italic underlined, SEQ ID NO: 37
ATGGCCTTACCAGTGACCGCCTTGCTCCTGCCGCTGGCCTTGCTGCTCCACGCCGCCAGG
CCGAGCCAGTTCCGGGTGTCGCCGCTGGATCGGACCTGGAACCTGGGCGAGACAGTGGA
GCTGAAGTGCCAGGTGCTGCTGTCCAACCCGACGTCGGGCTGCTCGTGGCTCTTCCAGCC
GCGCGGCGCCGCCGCCAGTCCCACCTTCCTCCTATACCTCTCCCAAAACAAGCCCAAGGC
GGCCGAGGGGCTGGACACCCAGCGGTTCTCGGGCAAGAGGTTGGGGGACACCTTCGTCC
TCACCCTGAGCGACTTCCGCCGAGAGAACGAGGGCTACTATTTCTGCTCGGCCCTGAGCA
ACTCCATCATGTACTTCAGCCACTTCGTGCCGGTCTTCCTGCCAGCGAAGCCCACCACGA
CGCCAGCGCCGCGACCACCAACACCGGCGCCCACCATCGCGTCGCAGCCCCTGTCCCTGC
GCCCAGAGGCGTGCCGGCCAGCGGCGGGGGGCGCAGTGCACACGAGGGGGCTGGACTT
CGCCTGTGATATCTACATCTGGGCGCCCTTGGCCGGGACTTGTGGGGTCCTTCTCCTGTCA
CTGGTTATCACCCTTTACTGCAACCACAGGAACCGAAGACGTGTTTGCAAATGTCCCCGG
CCTGTGGTCAAATCGGGAGACAAGCCCAGCCTTTCGGCGAGATACGTCGGTTCAAGAGC
TAAAAGAAGTGGTAGTGGTGCCCCTGTGA, SEQ ID NO: 38; (CD8α) nucleic acid sequence
VLLSNPTSG, CD8α CDR1, SEQ ID NO: 39
YLSQNKPK, CD8α CDR2, SEQ ID NO: 40
LSNSIM, CD8α CDR3, SEQ ID NO: 41

1. A method of manufacturing modified T cells comprising the steps of:
(a) activating an isolated population of T cells,
(b) culturing the T cells,
(c) modifying the T cells to express at least one heterologous T cell receptor (TCR) or chimeric antigen receptor (CAR), preferably by transducing the T cells with a nucleic acid or vector encoding the at least one heterologous T cell receptor (TCR) or chimeric antigen receptor (CAR),
(d) adding an inhibitor of AKT (AKT inhibitor) to the modified T cells,
(e) culturing the modified T cell population to expand and/or proliferate the cells or cell population,
(f) optionally harvesting and/or cryopreserving the modified T cell population.

2. The method according to claim 1 wherein the T cells are enriched for CD3+ fraction.

3. The method according to claim 1 wherein the activation stimulates the population of T cells to proliferate.

4. The method according to claim 3 wherein the activation is by addition of anti-CD3 antibody or antigen binding fragment thereof and/or anti-CD28 antibody or antigen binding fragment thereof, optionally attached to a removable bead.

5. The method according to claim 1, wherein modification is performed prior to or simultaneously with activation.

6. The method according to claim 1, wherein modification is performed after activation, preferably 18-26 hours after activation.

7. The method according to claim 1, wherein the AKT inhibitor is added after modification, preferably 17-24 hours after modification.

8. The method according to claim 1, wherein the AKT inhibitor is selected from the group consisting of: an allosteric inhibitor, a competitive ATP inhibitor, an inhibitor of interaction between AKT and the phospholipids, an inhibitor of phosphorylation of molecules downstream of AKT preferably of phosphorylation of PRAS 40, Ribosomal S6, TSC2.

9. The method according to claim 8, wherein the allosteric inhibitor is selected from, ARQ092, ARQ751, BAY1125976, or MK-2206.

10. The method according to claim 8, wherein the competitive ATP inhibitor is selected from, Afuresertib (GSK2110183), GSK2141795, GSK690693, Ipatasertib (GDC-0068), LY2780301, Triciribine (TCN-PM; VD-0002), AZD5363, or CCT128930

11. The method according to claim 8, wherein the inhibitor of the interaction between AKT and the phospholipids is Perifosine (D-21266, KRX0401).

12. The method according to claim 1, wherein the AKT inhibitor is added at a concentration of between 0.10 uM and 10 uM, preferably 0.5 uM.

13. The method according to claim 1, wherein the modified T cells or population of T cells produced in the presence of the AKT inhibitor have an increased or higher relative proportion of any one or more of:
(a) T cells expressing both CD45RA+, CCR7+,
(b) T cells that are CD45RO−, CCR7+, CD45RA+, CD62L+ (L-selectin), CD27+, CD28+ and IL-7Rα+,
(c) T cells that are T_{SCM} cells (stem memory T cells),
(d) T cells with a memory phenotype, preferably CD8 memory phenotype, in comparison to
(i) a population of T cells or modified T cells produced in the absence of AKT inhibitor, or
(ii) a population of T cells or modified T cells produced the presence of AKT inhibitor, wherein the AKT inhibitor is added prior to modification and/or less than or equal to 24 hours after activation.

14. The method according to claim 1, wherein the modified population of T cells produced in the presence of the AKT inhibitor have improved or increased level of any one or more of:
(a) persistence in-vivo, preferably persistence measured over 8 to 14 days post infusion in-vivo,
(b) expansion from seeding to harvest,
(c) durability of response or durable response rate (DRR),
(d) antigen induced interferon gamma production,
(e) T cell survival or lifespan or percentage viability,
(f) T cell effector function, preferably cytotoxicity,
in comparison to
(i) a population of T cells or modified T cells produced in the absence of AKT inhibitor, or
(ii) a population of T cells or modified T cells produced the presence of AKT inhibitor, wherein the AKT inhibitor is added prior to transduction and/or less than or equal to 24 hours after stimulation.

15. A population of modified T cells produced according to the method of claim 1, or a composition comprising the population and a physiologically acceptable excipient.

16. (canceled)

17. A method of adoptive therapy or for treating cancer or tumor, comprising administering to a patient in need thereof of population of modified T cells or composition according to claim 15.

18. (canceled)

19. A kit comprising a population of modified T cells produced according to the method of claim 1 and a package insert comprising instructions for using the T cells to treat or delay the progression of cancer and/or tumour in a subject.


SEQUENCE LISTING (42 sequences)

SEQ ID NO: 1 - protein, 10 aa, unidentified
  REGION 1..10: note = source = /note="Description of Unknown: MAGE A4 sequence"
GVYDGREHTV

SEQ ID NO: 2 - protein, 9 aa, Homo sapiens
FMNKFIYEI

SEQ ID NO: 3 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 4 - protein, 282 aa, synthetic construct
  REGION 1..282: note = source = /note="Description of Artificial Sequence: Synthetic polypeptide"
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 5 - DNA, 846 bp, synthetic construct
  misc_feature 1..846: note = source = /note="Description of Artificial Sequence: Synthetic polynucleotide"
  source 1..846: mol_type = other DNA
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagaga

SEQ ID NO: 6 - protein, 311 aa, synthetic construct
  REGION 1..311: note = source = /note="Description of Artificial Sequence: Synthetic polypeptide"
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 7 - DNA, 933 bp, synthetic construct
  misc_feature 1..933: note = source = /note="Description of Artificial Sequence: Synthetic polynucleotide"
  source 1..933: mol_type = other DNA
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 8 - protein, 136 aa, synthetic construct
  REGION 1..136: note = source = /note="Description of Artificial Sequence: Synthetic polypeptide"
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPD

SEQ ID NO: 9 - protein, 133 aa, synthetic construct
  REGION 1..133: note = source = /note="Description of Artificial Sequence: Synthetic polypeptide"
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLE

SEQ ID NO: 10 - protein, 6 aa, synthetic construct
  REGION 1..6: note = source = /note="Description of Artificial Sequence: Synthetic peptide"
VSPFSN

SEQ ID NO: 11 - protein, 6 aa, synthetic construct
  REGION 1..6: note = source = /note="Description of Artificial Sequence: Synthetic peptide"
LTFSEN

SEQ ID NO: 12 - protein, 15 aa, synthetic construct
  REGION 1..15: note = source = /note="Description of Artificial Sequence: Synthetic peptide"
CVVSGGTDSWGKLQF

SEQ ID NO: 13 - protein, 5 aa, synthetic construct
  REGION 1..5: note = source = /note="Description of Artificial Sequence: Synthetic peptide"
KGHDR

SEQ ID NO: 14 - protein, 6 aa, synthetic construct
  REGION 1..6: note = source = /note="Description of Artificial Sequence: Synthetic peptide"
SFDVKD

SEQ ID NO: 15 - protein, 14 aa, synthetic construct
  REGION 1..14: note = source = /note="Description of Artificial Sequence: Synthetic peptide"
CATSGQGAYEEQFF

SEQ ID NO: 16 - protein, 205 aa, synthetic construct
  REGION 1..205: note = source = /note="Description of Artificial Sequence: Synthetic polypeptide"
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 17 - DNA, 615 bp, synthetic construct
  misc_feature 1..615: note = source = /note="Description of Artificial Sequence: Synthetic polynucleotide"
  source 1..615: mol_type = other DNA
caaaaagaagttgagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaatagtgattccgggtatgcactcaacttcggcaaaggcacctcgctgttggtcacaccccatatccagaaccctgaccctgccgtgtaccagctgagagactctaagtcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcc

SEQ ID NO: 18 - protein, 242 aa, synthetic construct
  REGION 1..242: note = source = /note="Description of Artificial Sequence: Synthetic polypeptide"
DSGVTQTPKHLITATGQRVTLRCSPRSGDLSVYWYQQSLDQGLQFLIQYYNGEERAKGNILERFSAQQFPDLHSELNLSSLELGDSALYFCASSLGGESEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 19 - DNA, 726 bp, synthetic construct
  misc_feature 1..726: note = source = /note="Description of Artificial Sequence: Synthetic polynucleotide"
  source 1..726: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcctcgggggggaatctgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagac

SEQ ID NO: 20 - protein, 205 aa, synthetic construct
  REGION 1..205: note = source = /note="Description of Artificial Sequence: Synthetic polypeptide"
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 21 - protein, 242 aa, synthetic construct; identical to SEQ ID NO: 18
  REGION 1..242: note = source = /note="Description of Artificial Sequence: Synthetic polypeptide"

SEQ ID NO: 22 - protein, 6 aa, synthetic construct
  REGION 1..6: note = source = /note="Description of Artificial Sequence: Synthetic peptide"
DRGSQS

SEQ ID NO: 23 - protein, 6 aa, synthetic construct
  REGION 1..6: note = source = /note="Description of Artificial Sequence: Synthetic peptide"
IYSNGD

SEQ ID NO: 24 - protein, 12 aa, synthetic construct
  REGION 1..12: note = source = /note="Description of Artificial Sequence: Synthetic peptide"
AVNSDSGYALNF

SEQ ID NO: 25 - protein, 5 aa, synthetic construct
  REGION 1..5: note = source = /note="Description of Artificial Sequence: Synthetic peptide"
SGDLS

SEQ ID NO: 26 - protein, 6 aa, synthetic construct
  REGION 1..6: note = source = /note="Description of Artificial Sequence: Synthetic peptide"
YYNGEE

SEQ ID NO: 27 - protein, 11 aa, synthetic construct
  REGION 1..11: note = source = /note="Description of Artificial Sequence: Synthetic peptide"
ASSLGGESEQY

SEQ ID NO: 28 - protein, 6 aa, synthetic construct
  REGION 1..6: note = source = /note="Description of Artificial Sequence: Synthetic peptide"
DRGSQA

SEQ ID NO: 29 - protein, 12 aa, synthetic construct
  REGION 1..12: note = source = /note="Description of Artificial Sequence: Synthetic peptide"
AVNSDSSYALNF

SEQ ID NO: 30 - protein, 12 aa, synthetic construct
  REGION 1..12: note = source = /note="Description of Artificial Sequence: Synthetic peptide"
AVNSDSGVALNF

SEQ ID NO: 31 - protein, 12 aa, synthetic construct
  REGION 1..12: note = source = /note="Description of Artificial Sequence: Synthetic peptide"
AVNSQSGYALNF

SEQ ID NO: 32 - protein, 12 aa, synthetic construct
  REGION 1..12: note = source = /note="Description of Artificial Sequence: Synthetic peptide"
AVNSQSGYSLNF

SEQ ID NO: 33 - protein, 12 aa, synthetic construct
  REGION 1..12: note = source = /note="Description of Artificial Sequence: Synthetic peptide"
AVNSQNGYALNF

SEQ ID NO: 34 - protein, 6 aa, synthetic construct
  REGION 1..6: note = source = /note="Description of Artificial Sequence: Synthetic peptide"
DRGSFS

SEQ ID NO: 35 - protein, 6 aa, synthetic construct
  REGION 1..6: note = source = /note="Description of Artificial Sequence: Synthetic peptide"
DRGSYS

SEQ ID NO: 36 - protein, 12 aa, synthetic construct
  REGION 1..12: note = source = /note="Description of Artificial Sequence: Synthetic peptide"
AVNSQSSYALNF

SEQ ID NO: 37 - protein, 235 aa, synthetic construct
  REGION 1..235: note = source = /note="Description of Artificial Sequence: Synthetic polypeptide"
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 38 - DNA, 745 bp, synthetic construct
  misc_feature 1..745: note = source = /note="Description of Artificial Sequence: Synthetic polynucleotide"
  source 1..745: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcggttcaagagctaaaagaagtggtagtggtgcccctgtga

SEQ ID NO: 39 - protein, 9 aa, synthetic construct
  REGION 1..9: note = source = /note="Description of Artificial Sequence: Synthetic peptide"
VLLSNPTSG

SEQ ID NO: 40 - protein, 8 aa, synthetic construct
  REGION 1..8: note = source = /note="Description of Artificial Sequence: Synthetic peptide"
YLSQNKPK

SEQ ID NO: 41 - protein, 6 aa, synthetic construct
  REGION 1..6: note = source = /note="Description of Artificial Sequence: Synthetic peptide"
LSNSIM

SEQ ID NO: 42 - protein, 12 aa, synthetic construct
  REGION 1..12: note = source = /note="Description of Artificial Sequence: Synthetic peptide"
AVNSQSGVALNF